SEQUENCE LISTING (11 sequences)

SEQ ID NO: 1 - DNA, 24 bp, synthetic construct
  source 1..24: mol_type = other DNA
  misc_feature 1..24: note = Well-ID adaptor
cacgaccggtgctcgatttagggg

SEQ ID NO: 2 - DNA, 59 bp, synthetic construct
  source 1..59: mol_type = other DNA
  misc_feature 1..59: note = FW long primer 1
gagagactgacagcgtatcgcctccctcgcgccatcagcacgaccggtgctcgatttag

SEQ ID NO: 3 - DNA, 24 bp, synthetic construct
  source 1..24: mol_type = other DNA
  misc_feature 1..24: note = FW short primer1
gagagactgacagcgtatcgcctc

SEQ ID NO: 4 - DNA, 21 bp, synthetic construct
  source 1..21: mol_type = other DNA
  misc_feature 1..21: note = Kappa GSP1
cgattggagggcgttatccac

SEQ ID NO: 5 - DNA, 21 bp, synthetic construct
  source 1..21: mol_type = other DNA
  misc_feature 1..21: note = Lambda GSP1
tytgtgggacttccactgctc

SEQ ID NO: 6 - DNA, 23 bp, synthetic construct
  source 1..23: mol_type = other DNA
  misc_feature 1..23: note = Gamma GSP1
tcttgtccaccttggtgttgctg

SEQ ID NO: 7 - DNA, 18 bp, synthetic construct
  source 1..18: mol_type = other DNA
  misc_feature 1..18: note = FW primer2
cgtatcgcctccctcgcg

SEQ ID NO: 8 - DNA, 48 bp, synthetic construct
  source 1..48: mol_type = other DNA
  misc_feature 1..48: note = Kappa GSP long primer 2
ctatgcgccttgccagcccgctcagtcagatggcgggaagatgaagac

SEQ ID NO: 9 - DNA, 48 bp, synthetic construct
  source 1..48: mol_type = other DNA
  misc_feature 1..48: note = Lambda GSP long primer2
ctatgcgccttgccagcccgctcaggaggagggygggaacagagtgac

SEQ ID NO: 10 - DNA, 48 bp, synthetic construct
  source 1..48: mol_type = other DNA
  misc_feature 1..48: note = Gamma GSP long primer2
ctatgcgccttgccagcccgctcagggaagtagtccttgaccaggcag

SEQ ID NO: 11 - DNA, 20 bp, synthetic construct
  source 1..20: mol_type = other DNA
  misc_feature 1..20: note = RV primer2
ctatgcgcccttgccagccc

(19) United States
(12) Patent Application Publication
(10) Pub. No.: US 2026/0028593 A1
(43) Pub. Date: Jan. 29, 2026

(54) COMPOSITIONS AND METHODS FOR PRODUCING ANTIBODY-GENERATING IMMUNE ORGANOIDS

(71) Applicant: Parallel Biosystems, Inc., Boston, MA (US)

(72) Inventors: Robert M. DIFAZIO, Cambridge, MA (US); Juliana L. HILLIARD, Cambridge, MA (US)

(21) Appl. No.: 18/994,384

(22) PCT Filed: Jul. 14, 2023
(86) PCT No.: PCT/US2023/070281

§ 371 Date: Jan. 14, 2025

Related U.S. Application Data
(60) Provisional application No. 63/389,787, filed on Jul. 15, 2022.

(51) Int. Cl. C12N 5/071; C12N 5/0781; C12N 5/0783; C12N 5/0784

(52) U.S. Cl. CPC C12N 5/0697; C12N 5/0635; C12N 5/0636; C12N 5/0639; C12N 2502/1107; C12N 2502/1114; C12N 2502/1121; C12N 2513/00

(57) ABSTRACT

The present disclosure relates to the generation of antibody-producing, three dimensional, immune organoids. The disclosure also provides methods useful for producing such immune organoids.

### CROSS-REFERENCE TO RELATED APPLICATIONS

This application claims the benefit of and priority to U.S. Provisional Patent Application No. 63/389,787, filed Jul. 15, 2022, the disclosure of which is incorporated by reference herein in its entirety, including any drawings.

### FIELD

The present disclosure relates to the generation of antibody-producing immune organoids. For example, the present disclosure relates to three dimensional immune organoids capable of producing antibodies. The disclosure also provides methods useful for producing such immune organoids.

### BACKGROUND

Secondary immune organs, such as lymph node, tonsil and spleen, are highly structured tissues which dynamically change mechanical and biological functionality in response to antigens. Of particular importance is the activation of naïve B cells in these lymphoid tissues to form sub-anatomical structures, germinal center (GC), that program B cell conversion into antibody producing cells, which is critical for the development of immunological memory. To date, live animal models have mainly been used to understand immune cell development, functioning, and screening of immunotherapies against diseases, but such approaches are costly with long turnaround times. To address this problem, the successful generation of ex vivo organoids derived from secondary lymphoid tissue has been attempted by some in the field. However, such organoids have been limited by their source tissue (e.g. age of donor, type of tissue), requirement for stimulation, need for a high number of starting cells, limited types of cells present, limited function of antibodies produced, small number of organoids that can be made, among other problems.

The disclosure provided herein provides 3D in vitro immune organoids that that fully recapitulate the cellular complexity and critical functions of an in vivo secondary lymphoid organ, and can produce complete and robust humoral and cellular responses to antigen stimulation.

### SUMMARY

The present disclosure demonstrates the development of a new method for development of immune organoids from secondary lymphoid tissue which have the capacity to produce antibodies.

Provided herein, among others, includes a three dimensional immune organoid that includes a plurality of self-assembled primary immune cells obtained from one or more secondary lymphoid organs and a plurality of stem cells, wherein said the cells are CD34+, CD45RA−, ITGA3+, EPCR+, CD90+, CD73+, and CD105+.

In one embodiment, the one or more secondary lymphoid organs are from spleen, lymph node, Peyer's patch, and MALT.

In one embodiment, the immune organoid is a human immune organoid.

In one embodiment, the immune organoid further includes peripheral blood mononuclear cells.

In one embodiment, the plurality of immune cells is obtained from living patients, surgical resections, fine needle aspirates, biopsy, and deceased patients.

In one embodiment, the plurality of immune cells comprises B cells, T cells, plasmablasts, NK cells, monocytes, dendritic cells, macrophages and combinations thereof. In one embodiment, the B cells comprise one or more naïve B cells, pre-GC B cells, GC B cells, memory B cells, or a combination thereof. In one embodiment, the T cells comprise naïve CD4 T cells, memory CD4 T cells, T regulatory cells, T follicular helper cells, naïve CD8 cells, memory CD8 cells, gamma delta T cells, or a combination thereof. In one embodiment, the dendritic cells comprise conventional dendritic cells, plasmacytoid dendritic cells, myeloid dendritic cells, or a combination thereof.

In one embodiment, the plurality of primary immune cells further includes one or more stromal cells and fibroblastic reticular cells.

In one embodiment, the immune organoid is 8000 μm or less in diameter.

In one embodiment, the immune organoid comprises germinal centers and/or B/T cell zones. In one embodiment, the immune organoid is CXCR4⁺, CD83⁺, Ki67⁺, and IgD “. In one embodiment, the immune organoid is CD3” and CD20⁺.

In one embodiment, the immune organoid produces antibodies.

In one embodiment, the antibodies have full humoral functionality. In one embodiment, the antibodies bind human and non-human targets. In one embodiment, the human targets comprise proteins, sugars, and nucleic acid. In one embodiment, the non-human targets comprise infectious disease antigens, venoms, poisons, small molecules.

The present disclosure also provides a composition that includes a plurality of primary immune cells of which 100% self-assemble into three dimensional immune organoids within 24 hours.

In one embodiment, the plurality of immune cells are obtained from one or more secondary lymphoid organs. In one embodiment, the one or more secondary lymphoid organs are from spleen, lymph node, Peyer's patch, and MALT.

In one embodiment, the plurality of immune cells are human immune cells.

In one embodiment, the plurality of immune cells comprises 2×10⁶ or fewer cells.

In one embodiment, the composition further includes peripheral blood mononuclear cells.

In one embodiment, the plurality of immune cells is obtained from living patients, surgical resections, fine needle aspirates, biopsy, and deceased patients.

In one embodiment, the plurality of immune cells comprises B cells, T cells, plasmablasts, NK cells, monocytes, dendritic cells, macrophages and combinations thereof. In one embodiment, the plurality of primary immune cells further comprises one or more stromal cells, fibroblastic reticular cells, and stem cells.

The present disclosure further provides a method of producing multiple three dimensional immune organoids. The method includes (a) dissociating tissue from one or more secondary lymphoid organs to produce a plurality of single primary immune cells; (b) contacting 1×10⁶ or fewer of the plurality of single primary immune cells with a solid support; and (c) culturing the plurality of single primary immune cells for 24 hours to produce multiple three dimensional immune organoids, wherein the multiple immune organoids remain viable for at least 30 days.

In one embodiment, the method includes freezing the plurality of single primary immune cells prior to (b).

In one embodiment, the method includes thawing the plurality of single primary immune cells and treating with a ROCK inhibitor after freezing.

In one embodiment, the immune organoid is a human immune organoid.

In one embodiment, the secondary lymphoid organs are spleen, lymph node, Peyer's patch, and MALT. In one embodiment, the secondary lymphoid organs are obtained from living patients, surgical resections, fine needle aspirates, biopsy, and deceased patients.

In one embodiment, the plurality of primary immune cells includes B cells, T cells, plasmablasts, NK cells, monocytes, dendritic cells, macrophages and combinations thereof. In one embodiment, the plurality of primary immune cells further includes one or more stromal cells, fibroblastic reticular cells, and stem cells.

In one embodiment, the immune organoid is 8000 μm or less in diameter.

In one embodiment, the immune organoid is a 3 dimensional structure.

In one embodiment, the immune organoid comprises germinal centers and/or B/T cell zones. In one embodiment, the immune organoid is CXCR4⁺, CD83⁺, Ki67⁺, and IgD⁺. In one embodiment, the immune organoid is CD3” and CD20⁺.

In one embodiment, the immune organoid produces antibodies. In one embodiment, the antibodies have full humoral functionality. In one embodiment, the antibodies bind human and non-human targets. In one embodiment, the human targets comprise proteins, sugars, and nucleic acid. In one embodiment, the non-human targets comprise infectious disease antigens, venoms, poisons, small molecules.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B show that the stem cell population is represented in immune organoids. FIG. 1A shows representative flow cytometry staining of the stem cell populations contained in representative immune organoids in day 7 cultures. Cells shown are pre-gated on live, single, CD45+CD34+CD45RA-cells. Data shown across two donors. FIG. 1B shows quantification of the stem cell types in representative immune organoids in day 7 cultures. Cellular frequencies were determined by flow cytometry. Values plotted are across two different donors.

FIGS. 2A-2D show the diverse immune cell populations contained in a human immune organoid, including both innate and adaptive cells. FIG. 2A shows representative flow cytometry staining of B and T cells contained in representative immune organoids in day 7 cultures. Cells shown are pre-gated on live, single cells. Data shown across two donors. FIGS. 2B and 2C show representative flow cytometry staining of immune cell types contained in representative day 7 immune organoids, including dendritic cells, monocytes, macrophages, plasmablasts, and NK cells. Cells shown are pre-gated on live, single cells. Data shown across 2 donors. FIG. 2D shows quantification of a subset of immune cells represented in day 7 immune organoids. Cellular frequencies were determined by flow cytometry. Values plotted are across two different donors.

FIGS. 3A-3B show immune organoids undergo B cell differentiation upon stimulation. FIG. 3A shows representative images of flow cytometry staining demonstrating representative immune organoid B cell differentiation. All cells shown were previously gated on total B cells (CD3-CD19+CD45+). Phenotypes shown: CD38-CD27-naïve B cell, CD38-CD27+ memory B cell, CD38+CD27-pre-GC B cell, CD38+CD27+GC B cell. Plots from one representative donor shown. FIG. 3B shows quantification of B cells represented in day 7 immune organoids immunized with hemagglutinin protein. Cellular frequencies were determined by flow cytometry. Cells were previously gated on total B cells (CD3-CD19+CD45+).

FIGS. 4A-4E show immune organoids are composed of T cell subtypes. FIG. 4A shows representative images of flow cytometry staining demonstrating day 7 immune organoids consisting of T cells. All cells shown were previously gated on total T cells (CD19-CD3+). Cell types include CD4+ T cells and CD8+ T cells. Plots from 2 representative donors. FIGS. 4B and 4C show representative images of flow cytometry staining demonstrating immune organoids consist of T cell subtypes. All cells shown were previously gated on total T cells (CD19-CD3+) and then subsequently gated on CD4+ T cells and CD8+ T cells for their respective Naive and Memory T cell populations. Phenotypes shown: CD4+ CCR7+CD45RA+ Naive CD4 T cells, Naive CD8 T cells, CD4+ CCR7+CD45RA+, CD4+CD45RA-CD45RO+ Memory CD4 T cells, CD8+CD45RA-CD45RO+ Memory CD8 T cells. Plots are from 2 representative donors.

FIGS. 4D-4E show representative images of flow cytometry staining demonstrating immune organoids consist of additional T cell subtypes. All cells shown were previously gated on total T cells (CD19-CD3+). Cell types shown: CD3+CD4+CD25+ Tregs, CD3+CXCR5+CD25+ T follicular helper cells, CD3+CD27+gamma delta T cells. Plots are from 2 representative donors.

FIGS. 5A-5B show immune organoids are composed of other immune cell types including dendritic cells, stromal cells, and fibroblastic reticular cells. FIG. 5A shows representative images of flow cytometry staining demonstrating immune organoids consisting of dendritic cells (DCs). Cell types include myeloid DCs (CD14+CD11c+), plasmacytoid DCs (CD123+), conventional DCs (CD11b+CD45+), and CD14+DCs (CD14+CD11c+). Plots are from day 7 immune organoids across 2 representative donors. FIG. 5B shows representative images of flow cytometry staining demonstrating immune organoid cellular composition at day 7. Cell types include stromal cells (CD45−) and fibroblastic reticular cells (CD31+PDPN+). Plots are from representative immune organoids across 2 representative donors.

FIGS. 6A-6C show size of immune organoids. FIG. 6A shows representative brightfield images of day 14 immune organoids. Blue line indicates the area of a subset of representative organoids, with which diameter for each organoid was calculated. FIG. 6B shows longitudinal immune organoid diameter calculated from area of organoids ranging from day 1 to day 21. Median and error bars shown (n=4). FIG. 6C shows live cell counts of immune organoids at day 7 with cell counts ranging from 120,000 cells to 2,000,000 cells.

FIGS. 7A-7B show immune organoids form germinal centers consistent with lymph node function. FIG. 7A shows representative brightfield images of day 14 immune organoids stimulated with Hepatitis B vaccines. Lighter structures in the organoids outlined in red are consistent with germinal center morphology. FIG. 7B shows confocal microscopy images of germinal centers in the D14 immune organoids with B (CD20) and T cell (CD3) organization, plasmablasts (CD138), BCL6+ cells, and PD1+ cells.

FIGS. 8A-8G show immune organoids have cells consistent with germinal center function. FIG. 8A shows representative images of flow cytometry staining demonstrating immune organoids consisting of B and T cell zones. Cells shown CD19+ B cells and CD3+ T cells. Plots are from representative day 14 immune organoids. FIG. 8B shows representative images of flow cytometry staining demonstrating immune organoids consisting of germinal center B cells. Cells shown are gated on CD19+, CD3−, CD45+ cells and germinal center B cells are CD27+CD38+. Plots are from four representative day 14 immune organoids. FIGS. 8C-8F show representative images of flow cytometry staining demonstrating day 14 immune organoids consist of cells found in germinal centers including CD83+, Ki67+ B cells, Ki67+ T cells, IgD+ cells, and CXCR4+ cells. FIG. 8G shows quantification of germinal center cells in representative day 14 immune organoids. Cellular frequencies were determined by flow cytometry.

FIGS. 9A-9B show immune organoids are composed of both B and T cells that can be modulated upon various stimulation conditions. FIG. 9A shows quantification of immune organoid T cells in day 9 and day 16 immune organoids across unstimulated and 6 different stimulation conditions. N=4 organoids per stimulation condition on one representative donor. Cellular frequencies were determined by flow cytometry. Cells were previously gated on total T cells (CD3+CD19-CD45+). FIG. 9B shows quantification of immune organoid B cells in day 9 and day 16 immune organoids across unstimulated and 6 different stimulation conditions. N=4 organoids per stimulation condition on one representative donor. Cellular frequencies were determined by flow cytometry. Cells were previously gated on total B cells (CD3-CD19+CD45+).

FIGS. 10A-10D show immune organoids undergo a full adaptive immune response. FIG. 10A shows representative images of flow cytometry staining demonstrating Immune organoids undergoing plasmablast differentiation upon vaccination. Plots are from representative DO and D14 vaccinated immune organoids. All cells shown were previously gated on total B cells (CD19+CD3-CD45+). Phenotypes shown: CD38-CD27-naïve B cell, CD38-CD27+ memory B cell, CD38+CD27-pre-GC B cell, CD38+CD27+GC B cell, CD38+CD27+Plasmablasts. Plots from one representative donor shown. FIG. 10B shows antigen-specific IgG antibodies from day 4, day 8, day 11, day 15 immune organoids across 12 different stimulation conditions. Data shown from one representative donor. FIG. 10C shows antigen-specific IgM antibodies from day 4, day 8, day 11, day 14 immune organoids stimulated twice with 6 different stimulation conditions. Plots from one representative donor shown. FIG. 10D shows antigen-specific IgG antibodies from day 4, day 8, day 11, day 14 immune organoids stimulated twice with 6 different stimulation conditions. Plots from one representative donor shown.

FIGS. 11A-11D show immune organoids can break tolerance to generate antibodies against human targets. FIG. 11A shows influenza-specific IgM and IgG antibodies from day 11 immune organoids from unstimulated control organoids and 12 different influenza hemagglutinin protein stimulation conditions. Plots from one representative donor shown. FIG. 11B shows SARS-COV-2-specific IgM and IgG antibodies from day 11 immune organoids from unstimulated control organoids and 12 different SARS-COV-2 spike protein stimulation conditions. Plots from one representative donor shown. FIG. 11C shows Myelin-specific IgM and IgG antibodies from day 11 immune organoids from unstimulated control organoids and 8 different myelin protein stimulation conditions. Plots from one representative donor shown. FIG. 11D shows NK cell-specific IgM and IgG antibodies from day 11 immune organoids from unstimulated control organoids and 12 different NK cell protein stimulation conditions. Plots from one representative donor shown.

FIGS. 12A-B show immune organoids form within 24 hours and remain viable for at least 30 days. FIG. 12A shows representative brightfield images of day 1 immune organoids demonstrating full formation after 24 hours. FIG. 12B shows longitudinal brightfield images of representative immune organoids at day 1, day 3, day 4, day 11, day 14, day 17, day 21, day 24, day 28 immune organoids.

FIG. 13 shows immune organoids can also be cultured at the air-liquid-interface. Representative brightfield images of day 2 and day 4 air-liquid-interface (ALI) immune organoids as an alternative approach to culturing immune organoids are shown.

### DETAILED DESCRIPTION

The present disclosure provides, among others, three dimensional immune organoids that include a plurality of self-assembled primary immune cells obtained from one or more secondary lymphoid organs and a plurality of stem cells, wherein the stem cells are CD34+, CD45RA−, ITGA3+, EPCR+, CD90+, CD73+, and CD105+. These immune organoids address the problem with past attempts at immune organoid formation, due to the difficulty of producing organoids containing a full immune cell repertoire as well as the inability to produce a large number of organoids that can also generate an immune response against many antigens. Provided herein are, inter alia, three dimensional immune organoids produced with superior charactersitics which are suitable for generating antibodies against a wide variety of human targets. Some embodiments of the disclosure relate a composition including a plurality of primary immune cells of which 100% self-assemble into three-dimensional immune organoids within 24 hours. Further provided methods useful for producing multiple three dimensional immune organoids having the properties described above.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definition

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1−3 articles refers to groups having 1, 2, or 3 articles. Similarly, a group having 1−5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Although features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

### COMPOSITIONS OF THE DISCLOSURE

As described in greater detail below, one aspect of the present disclosure relates to a three dimensional immune organoid that includes a plurality of self-assembled primary immune cells obtained from one or more secondary lymphoid organs and a plurality of stem cells, wherein the stem cells are CD34+, CD45RA−, ITGA3+, EPCR+, CD90+, CD73+, and CD105+. Also provided are compositions that include a plurality of primary immune cells of which 100% self-assemble into three dimensional immune organoids within 24 hours.

### Immune Organoids

As described in greater detail below, one aspect of the present disclosure relates to a three dimensional immune organoid that includes a plurality of self-assembled primary immune cells obtained from one or more secondary lymphoid organs and a plurality of stem cells, wherein the stem cells are CD34+, CD45RA−, ITGA3+, EPCR+, CD90+, CD73+, and CD105+.

As used herein, a “three dimensional immune organoid” can be a composition of live immune cells, arranged in a three-dimensional or multi-layered configuration (as opposed to a monolayer).

In some embodiments, the immune organoid is produced ex vivo. A person of ordinary skill in the art would readily appreciate that the immune organoids described herein are also non-naturally occurring.

An organoid, in general, is an artificial construct created in vitro to mimic or resemble the functionality and/or histological structure of an organ, tissue, or a portion thereof. An organoid, as used herein, can be a cellular structure obtained by expansion of immune cells and stem cells, and consisting of tissue-specific cell types that self-organize. In the present disclosure, the term “organoid” can be used to refer to normal (e.g. non-tumour) organoids. An organoid can comprise one or more (e.g., 1, 2, 3, 4, or more) differentiated cell type(s) depending upon the particular tissue and/or organ being modeled or emulated.

### Immune Cells

The immune cells comprising the three dimensional immune organoid of the present disclosure are derived from one or more secondary lymphoid organs. As used herein, “derived from” generally refers to the source of primary cells that form the organoid. In some embodiments, “derived from one or more secondary lymphoid organs” can mean that the organoids are formed without any passage of the primary cells from the secondary lymphoid organs. In some embodiments, “derived from one or more secondary lymphoid organs” can mean that the organoids are formed after 1 passage of the primary cells. In some embodiments, “derived from one or more secondary lymphoid organs” can mean that the organoids are formed after more than 1 passage of the primary cells. A secondary lymphoid organ is a site where an adaptive immune response is initiated and lymphocytes are maintained. Exemplary secondary lymphoid organs include, lymph nodes (LNs), spleen, Peyer's patches (PPs), and mucosal associated lymphoid tissue (MALT), adenoids, and tonsils. In some embodiments, the primary immune cells are obtained from spleen, lymph node, Peyer's patch, and/or MALT.

The secondary lymphoid tissue can be obtained from a mammal (i.e., donor or patient) such as human, dog, cat, rabbit, monkey, chimpanzee, cow, pig, or goat. The secondary lymphoid tissue can be taken directly from living patients, surgical resections, fine needle aspirates, biopsy or deceased patients. In some embodiments, the secondary lymphoid tissue is obtained from a human and thereby results in a human immune organoid.

In some embodiments, the secondary lymphoid tissue is obtained and dissociated mechanically, enzymatically, or both. In some embodiments, the secondary lymphoid tissue is dissociated with a proteolytic and/or collagenolytic enzyme. In some embodiments, the secondary lymphoid tissue is enzymatically dissociated with Accutase (StemCell Technologies), Accumax (StemCell Technologies), trypsin, trypsin/EDTA, collagenase, dispase, TrypLE Express (Thermo Fisher), TrypLE Select (Thermo Fisher), or any combination thereof. In some embodiments, the secondary lymphoid tissue is mechanically dissociated by trituration, for example, with a pipette. In some embodiments, the single cell suspension of resulting cells is filtered to remove any non-dissociated cell masses.

The primary immune cells derived from the secondary lymphoid tissue can be any cell of hematopoietic origin that is functionally involved in the initiation and/or execution of innate and/or adaptive immune response, such as typically CD3 or CD4 positive cells. Exemplary types of primary immune cells include, without limitation, a dendritic cell, killer dendritic cell, a mast cell, a NK-cell, a plasmablast, a macrophage, a B-cell or a T-cell. In some embodiments, the plurality of immune cells includes B cells, T cells, plasmablasts, NK cells, monocytes, dendritic cells, macrophages and combinations thereof.

In some embodiments, the B cells comprise one or more naïve B cells, pre-GC B cells, GC B cells, memory B cells, or a combination thereof. In some embodiments, the B cells comprise primarily naïve B cells. In some embodiments, the B cells undergo B cell differentiation upon stimulation to yield pre-GC B cells, GC B cells, memory B cells, or a combination thereof. In some embodiments, the naïve B cells are CD38-CD27−. In some embodiments, the memory B cells are CD38-CD27+. In some embodiments, the pre-GC B cells are CD38+CD27−. In some embodiments, the GC B cells are CD38+CD27+.

In some embodiments, the T cells comprise naïve CD4 T cells, memory CD4 T cells, T regulatory cells, T follicular helper cells, naïve CD8 cells, memory CD8 cells, gamma delta T cells, or a combination thereof. In some embodiments, the naïve CD4 T cells are CD4+CCR7+CD45RA+. In some embodiments, the naïve CD8 T cells are CD8+CD45RA-CD27+. In some embodiments, the memory CD4 T cells are CD4+CCR7+CD45RA+, CD4+CD45RA-CD45RO+. In some embodiments, the memory CD8 T cells are CD8+CD45RA-CD45RO+. In some embodiments, the T regulatory cells are CD3+CD4+CD25+. In some embodiments, the T follicular helper cells are CD3+CXCR5+CD25+. In some embodiments, the gamma delta T cells are CD3+CD27+.

In some embodiments, the dendritic cells comprise conventional dendritic cells, plasmacytoid dendritic cells, myeloid dendritic cells, or a combination thereof. In some embodiments, the dendritic cells are CD45+CD11b+dendritic cells.

In some embodiments, the monocytes are CD14+CD11b+ monocytes.

In some embodiments, the macrophages are CD14+macrophages.

In some embodiments, the plasmablasts are CD38+CD27+plasmablasts.

In some embodiments, the NK cells are CD56+NK cells.

In some embodiments, the immune organoid as described herein produces and/or modifies T cells. By way of example, new T cell phenotype changes can occur in the immune organoid such as, for example, an increase in T helper cells. In some embodiments, new cytotoxic T cells and/or memory T cells can be produced in the immune organoid described herein. Such phenotypic changes can be identified using assays including, without limitation, flow cytometry to detect identifying markers on the T cells. Cytoxic T cells can be further identified, for example, using a T cell Cytotoxicity Assay as described in the Examples below.

In some embodiments, the plurality of immune cells present in an immune organoid of the present disclosure can be an amount of about 1%, 5%, 10%, 25%, 50% to about 55%, 60%, 75%, 80%, 90%, or 95%, of the total number of cells present in the organoid. In some embodiments, an organoid of the present disclosure comprises primary immune cells in an amount of about 1%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%, of the total number of cells present in the organoid. If an organoid of the present dislcosure comprises primary immune cells in an amount less than 100% of the total number of cells present in the organoid, then stem cells, stromal cells, and fibroblastic reticular cells in any suitable amount can make up the remaining number/percentage of cells.

In some embodiments, B cells are present in an immune organoid of the present disclosure in an amount of about 1%, 5%, 10%, 15%, or 20%. In some embodiments, T cells are present in an immune organoid of the present disclosure in an amount of about 1%, 5%, 10% or 15%. In some embodiments, NK cells are present in an immune organoid of the present disclosure in an amount of about 1%, 5%, 10% or 15%. In some embodiments, macrophages are present in an immune organoid of the present disclosure in an amount of about 1%, 2%, 3%, 4%, or 5%. In some embodiments, monocytes are present in an immune organoid of the present disclosure in an amount of about 1%, 2%, 3%, 4%, or 5%. In some embodiments, dendritic cells are present in an immune organoid of the present disclosure in an amount of about 1%, 2%, 3%, 4%, or 5%. In some embodiments, plasmablasts are present in an immune organoid of the present disclosure in an amount of about 1%, 2%, 3%, 4%, or 5%.

### Stem Cells

As described above, the immune organoid also includes a plurality of stem cells. Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny. The stem cells of the immune organoid described herein are identified as CD34+, CD45RA−, ITGA3+, EPCR+, CD90+, CD73+, and CD105+. Accordingly, the stem cells can include hematopoietic stem cells. Hematopoietic stem cells refer to a subset of multipotent stem cells that give rise to all the blood or immune cell types, including myeloid (monocytes and macrophages, neutrophils, basophils, eosinophils, erythrocytes, megakaryocytes/platelets, dendritic cells), and lymphoid lineages (T-cells, B-cells, NKT-cells, NK-cells). “Stem cells,” can refer to cells that retain the ability to renew themselves through mitotic cell division and can differentiate into a diverse range of specialized cell types. The stem stells can also include mesenchymal stem cells. Mesenchymal stem cells include stem cells that can be obtained, for example, from the bone marrow, peripheral blood, skin, hair root, muscle tissue, uterine endometrium, blood, cord blood, and primary cultures of various tissues. Mesenchymal stem cells can differentiate into all or several of osteocytes, chondrocytes and adipocytes.

Stem cells can be identified using flow cytometry and immunofluorescence as described in the Examples below. These methods are known in the art and involve the use of antibodies to detect the presence or absence of various protein markers on the surface of cells (e.g., CD34, CD45RA, ITGA3, EPCR, CD90, CD73, and CD105).

### Other Cell Types

In some embodiments, the immune organoid further includes peripheral blood mononuclear cells (PBMCs). PBMCs can be isolated from peripheral blood and identified as any blood cell with a round nucleus (i.e. lymphocytes, monocytes, natural killer cells (NK cells) or dendritic cells). The addition of PBMCs provides for an increase in the size of the immune repertoire of the organoid. By way of example, in some embodiments, the PBMCs are isolated from the same donor as the secondary lymphoid tissue. In some embodiments, the PBMCs are isolated from a different donor.

In other embodiments, the immune organoid further includes one or more stromal cells and fibroblastic reticular cells. A stromal cell is a type of cell that makes up certain types on connective tissue in the body. Fibroblastic reticular cells are stromal cells found in the secondary lymphoid lymphoid organs. The presence of both types of cells in the immune organoid described herein contribute to the deposition of extracellular matrix and the overall architecture of the three-dimensional immune organoid. By way of example, stromal cells can be identified by CD105+, CD29+, CD44+, CD90+, and CD45- and fibroblastic reticular cells can be identified by PDPN+ and CD31+using methods described in the Examples herein.

In some embodiments, stromal cells are present in an immune organoid of the present disclosure in an amount of about 0.5% 1%, 1.5%, or 2%. In some embodiments, fibroblastic reticular cells are present in an immune organoid of the present disclosure in an amount of about 1%, 2%. 3%, 4%, 5%, 6%, 7%, 8%, 9%, or 10%.

### Structure

In some embodiments, the immune organoid of the present disclosure has a diameter and/or largest dimension of about 8000 μm, 7500 μm, 7000 μm, 7000 μm, 6500 μm, 6000 μm, 5500 μm, 5000 μm, 4500 μm, 4000 μm, 3500 μm, 3000 μm, 2500 μm, 2000 μm, 1500 μm, 1000 μm, 500 μm, 250 μm, 100 μm, 50 μm or less. In some embodiments, the organoid may comprise about 1,500, 2,000, or 5,000 to about 10,000, 25,000, or 50,000 cells in total or about 1,000, 5,000, 10,000, or 50,000 to about 75,000, 100,000, 150,000, 250,000, 500,000, 750,000, 1,000,000, 50,000,000, or 100,000,000 cells in total. In some embodiments, an immune organoid of the present disclosure may comprise about 1, 2, or 5 million to about 10, 25, 50, or 100 million cells per mL. In some embodiments, an organoid of the present disclosure may comprise about 10 million cells per mL or 20 million cells per mL. In some embodiments, an organoid of the present disclosure may comprise about 5 or 10 million cells per mL to about 15 or 20 million cells per mL. An organoid of the present disclosure may be in any suitable three-dimensional shape or multi-layered shape. In some embodiments, an organoid of the present disclosure is in the form of a spheroid. In some embodiments, an organoid of the present disclosure may be self-organized in a suspension or medium.

In some embodiments, the immune organoid contains germinal centers and/or B/T cell zones. A germinal center (GC) is a sub-anatomical structure that programs B cell conversion into antibody producing cells. Within the GC, B cells undergo somatic mutation of the genes encoding their B cell receptors which, following successful selection, can lead to the emergence of B cell clones that bind antigen with high affinity. As described in Stebegg et al., “Regulation of the Germinal Center Response,” Front. Immunol. 9 (2018), the GC is divided into two distinct compartments: the light zone and the dark zone. In some embodiments, the immune organoid of the present disclosure contains both a light zone and a dark zone. The dark zone (DZ) contains a network of CXCL12-producing reticular cells and is the site of GC B cell proliferation and somatic hypermutation (SHM). Centroblasts then follow a CXCL13 gradient to enter the light zone (LZ) as centrocytes through their expression of CXCR5. In the LZ, centrocytes capture antigen presented on follicular dendritic cells which they internalize, process and subsequently present to T follicular helper cells in order to undergo selection. This process is regulated by T follicular regulatory cells which are also present in the LZ. Upon receiving survival signals from Tfh cells, centrocytes re-enter the DZ for further rounds of proliferation and SHM after which they exit the GC as memory B cells or high-affinity antibody-secreting plasma cells. Thus, the presence of both the dark zone and the light zone in an immune organoid of the present disclosure can be identified using techniques including, without limitation, immunofluorescence as described in the Examples. In some embodiments, the germinal centers in the immune organoid are characterized as CXCR4⁺, CD83⁺, Ki67⁺, and IgD “. In some embodiments, the B and T cell zones in the germinal centers of the immune organoid are characterized as CD3” and CD20⁺.

### Antibody Production

In some embodiments, the immune organoid of the present disclosure can be capable of producing antibodies. Antibodies are proteins used by the immune system to identify and neutralize foreign objects such as pathogenic bacteria and viruses. The antibody recognizes a unique molecule of the pathogen, called an antigen. Antibodies can come in different varieties known as isotypes or classes. In placental mammals there are five antibody classes known as IgA, IgD, IgE, IgG, and IgM, which are further subdivided into subclasses such as IgAQ1, IgA2. Accordingly, the immune organoid of the present disclosure can be capable of producing IgA, IgD, IgE, IgG, IgM, and combinations thereof. The prefix “Ig” stands for immunoglobulin, while the suffix denotes the type of heavy chain the antibody contains: the heavy chain types α (alpha), γ (gamma), δ (delta), ε (epsilon), μ (mu) give rise to IgA, IgG, IgD, IgE, IgM, respectively. The antibody isotype of a B cell changes during cell development and activation.

Immature B cells, which have never been exposed to an antigen, express only the IgM isotype in a cell surface bound form. The B lymphocyte, in this ready-to-respond form, is known as a “naive B lymphocyte.” The naive B lymphocyte expresses both surface IgM and IgD. The co-expression of both of these immunoglobulin isotypes renders the B cell ready to respond to antigen. B cell activation follows engagement of the cell-bound antibody molecule with an antigen, causing the cell to divide and differentiate into an antibody-producing cell called a plasma cell. In this activated form, the B cell starts to produce antibody in a secreted form rather than a membrane-bound form. Some daughter cells of the activated B cells undergo isotype switching, a mechanism that causes the production of antibodies to change from IgM or IgD to the other antibody isotypes, IgE, IgA, or IgG, that have defined roles in the immune system. Accordingly, the immune cells of the immune organoid of the present disclosure can also encompass those that have undergone or will undergo the process of isotype switching.

Antibodies are critical for development of a humoral immune response in which antibodies are produced by B cells and are secreted into the blood and/or lymph in response to an antigenic stimulus. In a properly functioning immune response, the antibody binds specifically to antigens on the surface of cells (e.g., a pathogen), marking the cell for destruction by phagocytotic cells and/or complement-mediated mechanisms. Briefly, antibodies participate in several important functions including antibody dependent cellular cytotoxicity (ADCC), phagocytosis (opsonization), and complement-dependent cytoxicity (CDC), upon binding to antibody-bound target cell.

Thus, in some embodiments, the immune organoid has full humoral functionality. Specifically, in some emobdiments, antibodies produced by the immune organoid of the present disclosure can function in ADCC. ADCC is an in vitro or in vivo process where an antibody can bind to an antigen on a surface of a cell then engage with immune-effector cells via sequences within the antibody's Fc domain that in turn results their release of toxins that can kill bound cell. ADCC activity can be measured using methods known in the art including, without limitation, using in vitro methods as described in the Examples herein.

In some embodiments, antibodies produced by the immune organoid of the present disclosure can function in CDC. CDC refers to an in vitro or in vivo process where an antibody can bind to an antigen on a surface of an eukaryotic or prokaryotic cell then engage with the Clq protein via sequences within the antibody's Fc domain that in turn results in initiation of classical complement cascade that can kill bound cell. CDC activity can be measured using methods known in the art including, without limitation, using in vitro methods as described in the Examples herein.

In some embodiments, antibodies produced by the immune organoid of the present disclosure can function in opsonization. Opsonization is a process where an antibody can bind to an antigen on a surface of a cell then engage with immune cells via sequences within its Fc domain that in turn results in immune cells engulfing, consuming and ultimately killing antibody bound cell. Opsonization activity can be measured using methods known in the art including, without limitation, using in vitro methods as described in the Examples herein.

In some embodiments, the immune organoid as described herein exhibits at least one of ADCC, CDC, and opsonization activity. In some embodiments, the immune organoid as described herein exhibits at least two of ADCC, CDC, and opsonization activity. In some embodiments, the immune organoid as described herein exhibits all three of ADCC, CDC, and opsonization activity.

In some embodiments, the antibodies produced by the immune organoid of the present disclosure can also undergo the process of somatic hypermutation. Somatic hypermutation refers to a process of enhanced mutation of a gene, thought to require activation-induced cytidine deaminase (AID) and error-prone DNA repair. SHM was initially described from observations of the increased mutation of immunoglobulin gene regions encoding variable regions of the light and heavy chains in B lymphocytes following antigen stimulation. AID is discussed, for example, in Smith et al., Trends Genet. 20:224−227 (2004). The presence of Somatic hypermutation can be identified using methods including, without limitation, measurement of AID upregulation by, for example, quantitative PCR methods. B cell receptor sequencing can also be used to identify collections of mutations.

In some embodiments, the immune organoids described herein produce plasmablasts and antigen-specific antibodies against targets to which patient donors have been exposed (recall response) and to which patients are naïve.

In some embodiments, the immune organoids described herein produce autoimmune responses against self antigen.

In some embodiments, the antibodies produced by the immune organoids as described herein bind human and non-human targets. Exemplary human targets include, without limitation, proteins, sugars, and nucleic acid. Exemplary non-human targets can include, without limitation, infectious disease antigens, venoms, poisons, small molecules.

### COMPOSITIONS

As described in greater detail below, one aspect of the present disclosure relates to a plurality of primary immune cells of which 100% self-assemble into three-dimensional immune organoids within 24 hours.

As described above, the plurality of primary immune cells can be obtained from one or more secondary lymphoid organs. Secondary lymphoid organs useful in the compositions and methods of the present disclosure are also described above.

The secondary lymphoid tissue can be obtained from a mammal (i.e., donor or patient) such as human, dog, cat, rabbit, monkey, chimpanzee, cow, pig, or goat. The secondary lymphoid tissue can be taken directly from living patients, surgical resections, fine needle aspirates, biopsy or deceased patients. In some embodiments, the secondary lymphoid tissue is obtained from a human and thereby results in a human immune organoid.

In one embodiment, a plurality of immune cells obtained from one or more secondary lymphoid organoids from a single donor produces about 1000, about 100000, about 20000, about 30000, about 40000, about 50000 organoids. In some embodiments, the plurality of immune cells obtained from one or more secondary lymphoid organs from a single donor produces about 50000 organoids. This is in contrast to the approximate 25 organoids produced per donor using prior known techniques.

In some embodiments, the plurality of primary immune cells 100% self-assemble into three-dimensional immune organoids in 1, 2, 3, 4, 5, 6, 7, 8, 9, 10, 11, 12, 13, 14, 15, 16, 17, 18, 19, 20, 21, 22, 23, or 24 hours.

Type of immune cells that can be present in the plurality of immune cells are described above. The plurality of immune cells can comprise about 2×10⁶, 1.5×10⁶. 1×10⁶ 0.5×10⁶. 0.4×10⁶ 0.3×10⁶. 0.2×10⁶. 0.1×10⁶ or fewer total cells.

As described above, the inclusion of additional cell types are contemplated in the composition of the disclosure. Additional cell types include, without limitation, PBMCs, stromal cells, and fibroblastic reticular cells.

### METHODS OF THE DISCLOSURE

The present disclosure provides, among others, a method of producing multiple immune organoids. The method includes (a) dissociating tissue from one or more secondary lymphoid organs to produce a plurality of single primary immune cells; (b) contacting 1×10⁶ or fewer of the plurality of single primary immune cells with a solid support; and (c) culturing the plurality of single primary immune cells for 24 hours to produce multiple three dimensional immune organoids, wherein the multiple immune organoids remain viable for at least 30 days.

Secondary lymphoid tissue useful in the methods of the present disclosure are described above for the compositions. Briefly, the secondary lymphoid tissue can be obtained from a mammal (i.e., donor or patient) such as human, dog, cat, rabbit, monkey, chimpanzee, cow, pig, goat. The secondary lymphoid tissue can be taken directly from living patients, surgical resections, fine needle aspirates, biopsy or deceased patients. In some embodiments, the secondary lymphoid tissue is obtained from a human and thereby results in a human immune organoid.

Tissues can be dissociated by a variety of methods, including, without limitation, both mechanical and/or enzymatic treatments. For example, the tissue dissociation can be mechanical (shredded or sheared), or used with single or combined proteolytic enzymes such as matrix metalloproteases and/or neutral proteases such as collagenase, trypsin, dispase, LIBERASE (Boehringer Mannheim), Accumax. It can be dissociated by enzymatic digestion with Hyalronidase, and/or pepsin, or a combination of mechanical and enzymatic methods. Methods of tissue dissociation are also described above.

Using the methods described herein, the dissociation of the secondary lymphoid tissue produces a suspension of single primary immune cells. These primary immune cells can be any cell of hematopoietic origin that is functionally involved in the initiation and/or execution of innate and/or adaptive immune response, such as typically CD3 or CD4 positive cells. Exemplary types of primary immune cells include, without limitation, a dendritic cell, killer dendritic cell, a mast cell, a NK-cell, a plasmablast, a macrophage, a B-cell or a T-cell and combinations thereof.

In some embodiments, the method involves freezing the plurality of single primary immune cells prior to step (b).

In some embodiments, prior to step (c), frozen immune cells are thawed and treated with a ROCK inhibitor. ROCK is a serine/threonine kinase that functions as a target protein for Rho (there are three isoforms—there are RhoA, RhoB and RhoC). Exemplary ROCK inhibitors include, but are not limited to, antibodies to ROCK, dominant negative ROCK variants, and siRNA and antisense nucleic acids that suppress ROCK expression. Other exemplary ROCK inhibitors include, but are not limited to, thiazovivin, Y27632, Fasudil, AR122-86, Y27632H-1152, Y-30141, Wf-536, HA-1077, hydroxyl-HA.-1077, GSK2699962A, SB-772077-B, N-(4-pyridyl)-N′-(2,4,6-trichlorophenyl) urea, 3-(4-pyridyl)-1H-indole, and (R)-(+)-trans-N-(4-pyridyl)-4-(1-aminoethyl)-cyclohexanecarboxamide, pyrintegrin, and blebbistatin.

As described herein, 1×10⁶ or fewer single primary immune cells are contacted with a solid support. Solid support can include a reservoir and/or a plurality of reservoirs (e.g., wells of a well plate). The reservoir(s) may be any suitable reservoir or container that holds the organoid. In some embodiments, the reservoir is a well of a well plate such as, but not limited to, a well in a 6-well plate, a 12-well plate, a 24-well plate, a 48 well plate, a 96-well plate, a 384-well plate, transwells, ULA plates, flat-bottom plates, or V-bottom plates. The solid support can also include a pedestal for use in an air-liquid interface. An air-liquid interface can be the interface to which the primary immune cells are exposed to in the cultures described herein. The primary tissue may be mixed with a gel solution which is then poured over a layer of gel formed in a container with a lower semi-permeable support, e.g. a membrane. This container is placed in an outer container that contains the medium such that the gel containing the tissue in not submerged in the medium. The primary tissue is exposed to air from the top and to liquid medium from the bottom.

In certain embodiments, the cell culture medium in which the organoids are cultured comprises a serum-supplemented cell culture medium. Features of the the medium in which the cells are cultured can also include, for example, any growth factors or differentiation-inducing factors that may be present, and a supporting structure (such as a substrate on a solid surface) if present.

According to methods of the present disclosure, the plurality of single primary immune cells are cultured for 24 hours to produce multiple immune organoids, such that the multiple immune organoids remain viable for at least 30 days. In some embodiments, the multiple immune organoids remain viable for 30, 35, 40, 45, 50, 55, 60, 65, 70, 75, 80, 85, 90, 95, or 100 days. In some embodiments, about 1000, about 100000, about 20000, about 30000, about 40000, about 50000 organoids are produced using tissue from a single donor as described herein.

In some embodiments, the plurality of immune cells further comprises one or more stromal cells, fibroblastic reticular cells, and stem cells as described above for the compositions.

In some embodiments, using the method described herein, the multiple immune organoids have a diameter and/or largest dimension of about 8000 μm, 7500 μm, 7000 μm, 7000 μm, 6500 μm, 6000 μm, 5500 μm, 5000 μm, 4500 μm, 4000 μm, 3500 μm, 3000 μm, 2500 μm, 2000 μm, 1500 μm, 1000 μm, 500 μm, 250 μm, 100 μm, 50 μm or less.

In some embodiments, using the method provided herein, the multiple organoids also contain germinal centers and/or B/T cell zones as described above. In some embodiments, the multiple immune organoids are CXCR4⁺, CD83⁺, Ki67⁺, and IgD “. In some embodiments, the multiple immune organoids are CD3” and CD20⁺.

In some embodiments, using the method provided herein, the multiple immune organoids produce antibodies. Various antibody types and functions as well as resulting humoral immunity are described in detail above.

### EXAMPLES

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 1: Methods

Tissue Collection and freezing. Whole spleens and whole lymph nodes from, e.g., deceased donors (of all ages) or live donors (of all ages) are collected and sliced into segments by accredited clinicians, placed in hypothermosol+2× Pen/Strep, 1× Normocin and stored at 4C for shipment and until further processing. Hypothermosol medium is prepared and workspace is prepared. Cryovials are labeled with date, tissue type, batch ID number, name [link to cryovial sticker template]. Upon arrival in the lab, tissues are fully immersed in fresh hypothermosol+2×Pen/Strep, 1× Normocin and unprocessed segments are kept on ice (4° C.). Using a disposable scalpel, tissue is carefully dissected into 5 mm×5 mm×5 mm pieces and placed into 24 well plates for dissociation. Tissue is rinsed with 1 ml 0.5 mM EDTA and then incubated at 37° C. in 1 ml 0.5 mM EDTA for 1 hour (modulate and record change depending on results). Tissue is incubated with 1 ml Accumax at 37° C. for 1 hour, the reaction is stopped with 1 ml Complete Medium. The cells are counted using the NC-200 and live cell count and viability is recorded. Cells are centrifuged at 250×g for 10 minutes. Cells are aspirated and resuspended in 1 ml Cryostore at 10×10⁶ cells/mL per vial. Cells are then transferred to pre-barcoded cryovials. Vials are transferred to Mr. Frosty and stored at −80° C. overnight, and transferred to LN2 the next day. The barcode is recorded in LN2 inventory (LN2 number, hotel number, box number, and placement in box).

Organoid formation. Upon thaw, ROCK inhibitor (e.g., Y-27632) is added to Complete medium. Cryovials are partially submerged a in 37° C. bath and thawed until a sliver of ice remains. Cells are transferred from the cryovial to a 50 ml conical tube. 1 ml Complete medium is added dropwise, swirling between each drop until 9 ml Complete medium has been added. Spin at 200×g for 5 min. Supernatant is discarded and cells are resuspended in 2 ml of complete medium. The cells are counted using the NC-200 and the tubes are spun again while counting (200×g for 5 min). Resuspension volume is calculated to achieve 100 μl per well in the upper chamber (1×10⁶ to 2×10⁶ cells total). The complete medium is supplemented with 1 μg/ml BAFF and 200 μl supplemented medium is added per well of e.g., transwells, ULA plates, flat-bottom plates, V-bottom plates, or a pedestal. Stimulation is added directly to the culture prior to incubation 37° C. overnight. Organoids are supplemented with BAFF supplemented complete medium every 3 days.

Addition of PBMC's. Once formed, organoids are supplemented with adaptive cells from PBMCs.

Flow cytometry. Staining buffer is prepared (1× DPBS+0.5% BSA, or 1× DPBS+1% FBS) and a Flow Panel is designed using the Biolegend Spectra Analyzer tool (https://www.biolegend.com/en-us/spectra-analyzer). The organoid containing plate is removed out of the incubator and placed in a precleaned biosafety cabinet. The organoid is disrupted to create a single cell suspension. Cells are counted and and placed into flow tubes. 2 mL of staining buffer is added to each tube. Cells are centrifuged at 500 g for 5 minutes and resuspended in about 50 μL of staining buffer. The appropriate amount of primary antibody is added per manufacturer's directions and incubated for 30 minutes at 4C, protected from light. The sample is washed 2×with 2 mL of Flow Staining buffer, the supernatant is aspirated and cells are resuspended in residual buffer. Secondary antibody is added per manufacturer's instructions and incubated for 30 at 4C, protected from light. Sample is washed 2× with 2 mL of Flow Staining buffer, supernatant is aspirated, and testing sample is resuspended in 250 μL of staining buffer and unstained control in 350 μL. If needed, cells are be fixed at this point using 1% paraformaldehyde. Single-color compensation controls are prepared and sample is analyzed.

Immunofluorescence. Slides with sections are thawed and allowed to dry completely. Slides are submerged in PBS to remove OCT for 15 minutes at room temperature. Slides are placed in Sequenza clips while submerged in PBS. Clips are carefully placed with slides attached into the sequenza racks. PBS is run through the slides to ensure they are appropriately sealed and that no PBS runs through too quickly. Permeabilization and blocking, 150 μl of permeabilization buffer is added to each slide and incubated for 1 hr. Sections are washed with 150 μl of PBS 3 times for 5 minutes. 150 μl of blocking buffer is added to each section and incubated for 1 hour with the rack completely covered at room temperature.

Primary antibodies are prepared by diluting to their proper concentrations in the blocking buffer in appropriate volume. 150 μl of antibody solution is added to each section and incubated overnight completely covered at 4° C.

Secondary antibodies are prepared by diluting to their proper concentrations in the blocking buffer and protected from light using aluminum foil and kept at 4° C. DAPI is prepared by diluting to its proper concentration in PBS and protected from light using aluminum foil and keep at 4° C.

Slides are washed with PBS 3 times for 5 minutes, and 150 μl of antibody solution is added to each section and incubated for 2 hours completely covered at room temperature. Slides are then washed with PBS 3 times for 5 minutes. 150 μL of DAPI is added to each section and incubated for 10 minutes completely covered at room temperature. Slides are washed with PBS 3 times for 5 minutes.

ELISA. First the assay diluent (1% BSA in 1×PBS w/o+Mg and +Ca) and wash buffer (1×PBS+0.05% Tween-20) is prepared. The ELISA plate is coated using a coating buffer diluted from 5× ELISA coating buffer to 1× working solution in DI water. Columns “1” and “2” are coated with coating buffer mixed with IgG capture Ab at a dilution of 1:300 (100 μL/well dilution mix capture antibody). The remaining wells of plate are coated with coating buffer mixed with influenza A recombinant protein at a concentration of 0.1 ug/well (Seed 100 μL per well). The plate is covered with film and placed in shaker for 1 hr at 20−25C or covered with film and incubated overnight at 4C. The ELISA plate is blocked by washing with 300 μL of prepared wash buffer in each well. This is repeated until it is completed for a total of 4 times. 200 μL of Assay Diluent is added to each well of plate and placed on shaker at room temperature for a total of 1 hour.

While plates are blocking, all standards are prepared. The standard is diluted with assay diluent to a concentration of 1000 ng/mL. 500 μL of assay diluent is added to each tube. From the initial prepared standard (200 ng/mL) 500 μL of AD is added. This is now tube 1 and final concentration is 100 ng/mL. 2× serial dilutions are continued down to the final tube. One tube will contain just assay diluent.

The plate is removed from shaker washed for a total of 4 times. Prepared standards are added to wells in 100 μL duplicates, and sample dilution is determined. Assay diluent is added to all sample wells of plate based on sample dilution for a total volume of 100 μL/well. The plate is placed on shaker for 2 hours at room temperature.

For the addition of detection antibody-HRP, detection antibody dilution is prepared at a 1:100,000 dilution. The plate is removed from shaker and washed for a total of 4 times. 100 μL of prepared detection antibody solution is added to all wells of plate. The plate is covered with film and placed on shaker for 1 hour.

The next step is a 2TMB Substrate Incubation and Reaction STOP. TMB substrate is prepared and kept away from light. For one plate 5.5 mL of each solution is added. The plate is removed from the shaker washed with 300 μL of prepared wash buffer per each well for a total of 5 times. 100 μL of prepared TMB solution is added to each well of the plate. The plate is placed in the dark and monitored for color change. 100 μL of ELISA STOP solution or (2N H₂SO₄) is added to each well when color in 4th standard is developed.

Data is acquired on plate reader. For data analysis, duplicate absorbance values should be within 10% of each other. A standard curve is plotted for the IgG standard (known concentration) samples, and the average absorbance value minus (−) the blank value for each standard for each standard concentration is plotted on the vertical (Y) axis. The corresponding human IgG concentration is plotted the horizontal axis (X) that correlates with the absorbance values. This is used to extrapolate the concentration for the unknown sample.

Somatic hypermutation. Cells are harvested from day 0 and a comparative end point. Cells are washed with FACS buffer and incubated with biotinylated recombinant protein of interest at a concentration of 4 μg/mL, in the presence of Fc block. In addition, cells are incubated with fluorescently labeled antibody panels to determine B cell lineage (CD38+CD27+). +Protein B cells of GC or plasmablast phenotype are sorted out into 96-well plate. cDNA is collected and tagged from individual B cells with unique DNA barcodes and pooled by plate. Gene specific PCR is used to amplify immunoglobulin heavy and light change variable regions. Libraries are sent for sequencing, and the sequence is analyzed. A Fastq file is generated by demultiplexing using a MiSeq Reporter and is quality filtered. Paired reads are stitched and separated by well ID and consensus sequences. The well ID reads are clustered into operational taxonomic units. Operational taxonomic unit sequences are anlalyzed with IMGT High V-QUEST. Clonal families are defined by the same V and J gene usage and at least 70% amino acid identity in the CDR3 locos for both heavy and light chains.

Exemplary primer sequences used for the detection of somatic hypermutation are shown in Table 1 below.

TABLE 1

| Sequence | |
|---|---|
| Name | Sequence |
| Well-ID | CACGACCGGTGCTCGATTTAG(WELL-ID)GGG (SEQ ID NO: 1) |
| adaptor | |
| FW long | GAGAGACTGACAGCGTATCGCCTCCCTCGCGCCATCAG |
| primer 1 | (PLATE-ID)CACGACCGGTGCTCGATTTAG (SEQ ID NO: 2) |
| FW short | GAGAGACTGACAGCGTATCGCCTC (SEQ ID NO: 3) |
| primer1 | |
| Kappa GSP1 | CGATTGGAGGGCGTTATCCAC (SEQ ID NO: 4) |
| Lambda GSP1 | TYTGTGGGACTTCCACTGCTC (SEQ ID NO: 5) |
| Gamma GSP1 | TCTTGTCCACCTTGGTGTTGCTG (SEQ ID NO: 6) |
| FW primer2 | CGTATCGCCTCCCTCGCG (SEQ ID NO: 7) |
| Kappa GSP | CTATGCGCCTTGCCAGCCCGCTCAG(PLATE-ID) |
| long primer 2 | TCAGATGGCGGGAAGATGAAGAC (SEQ ID NO: 8) |
| Lambda GSP | CTATGCGCCTTGCCAGCCCGCTCAG(PLATE-ID) |
| long primer2 | GAGGAGGGYGGGAACAGAGTGAC (SEQ ID NO: 9) |
| Gamma GSP | CTATGCGCCTTGCCAGCCCGCTCAG(PLATE-ID) |
| long primer2 | GGAAGTAGTCCTTGACCAGGCAG (SEQ ID NO: 10) |
| RV primer2 | CTATGCGCCCTTGCCAGCCC (SEQ ID NO: 11) |

FW, forward; RV, reverse; GSP, gene-specific primer

Antibody function. ADCC. To prepare target cells, 2×10\hat{ }5 cells/ml are washed 3 times with 5 ml of RPMI 1640 containing 2 μg/ml of TPCK-trypsin. Media is removed and virus or pathogen is inoculated at a MOI2. The inoculum is allowed to adsorb for 60 min at 37° C. Cells are gently washed with 6 ml of media (RPMI1640) containing 2 μg/ml of TPCK trypsin without serum. 5 ml of media (RPMI1640) containing 2 μg/ml of TPCK trypsin with 2% calf serum is added to T-25 flasks. Cells are incubated for 48 hours at 37%. A sample of target cells is taken after incubation to assess the incidence of infected cells through the ability to produce hem-agglutination with Turkey RBCs and binding with polyclonal antibodies. 80−95% of cells should be infected. 2×10\hat{ }6 cells are washed with PBS twice. Cells are laealed with PKH67.

For preparation of effector cells (Isolation of PBMCs), PBMCs are thawed from the biobank. PBMCs are washed twice with PBS. The antibody-dependent cell-mediated cytotoxicity assays (ADCC) are dispensed using 5.0×10\hat{ }4 labeled target cells in 50 μl RPMI 1640 media in each well as per the layout given below in round-bottom 96 well plate in duplicate. 50 μl of antibodies are added to the wells as per layout and incubated for 15 min at 37° C. in CO₂ incubator. Unlabelled normal PBMC effector cells in 100 μl of RPMI 1640/0.5% Pen/Strep at a concentration of 2.5×10\hat{ }7 cells/ml are added to each well as per the layout. Cells re incubated for 2 h at 37° C. in CO₂ incubator. After 2 h, 1 μl of the fluorescent dead cell dye 7-amino-actinomycin-D (7-AAD) is added and incubated at 4° C. in dark for 20 min. Cells are analyzed on a flow cytometer and a total of 5,000 target cells are acquired. The percentage cell death is determined by software analysis of four identifiable cell populations, live effector cells (no dye), dead effector cells (7-AAD only), live target cells (PKH-67 only) and dead target cells (PKH-67 and 7-AAD).

Antibody function. CDC′. 20 μL of target cells are added at a concentration of 2×10⁵ cell/ml to each well of the 96-well assay plate. A 10-point titration curve of the test antibody is created using 1:2.5 serial dilutions beginning at 1 μg/ml. Each antibody dilution is added to the plate to start the reaction. The plate is placed on an orbital shaker for 30 seconds and then transferred to a 37° C./5% CO₂ incubator for 15 min to allow the cells to opsonize. Complement is diluted 1:18 in complete medium and added 25 μl to the appropriate wells. The plate is placed on an orbital shaker for an additional 30 seconds and placed in a 37° C./5% CO₂ incubator for 30 minutes. The plate is removed from the incubator and allowed to cool to room temperature for 15 minutes. 10 μl of lysis buffer is added to the lysis control wells. The plate is incubated on the benchtop at room temperature for 5 minutes. 125 μl of medium is added to each well. The plates are centrifuged for 1 minute at 750 RPM. 50 μl enzyme assay diluent is added to appropriate wells of white luminescence plate. 50 μl of the reaction supernatant is added to the wells containing assay diluent. 100 μl of 2× enzyme assay reagent is added to each diluted supernatant. 50 μl of 1× detection reagent is added to each diluted supernatant. The plates are shaken for 30 seconds and then immediately read on luminescence on the plate reader.

Antibody function. Antibody-Dependent Cell-Mediated Phagocytosis. Target cells are labeled with PKH67 and seeded at 25,000 per well into 96-well plates with 10% human AB serum. Serial dilution of antibodies of interest are performed to 8 half-log concentrations and added to target cells. Macrophages are added at 4:1 effector: target. Cells are centrifuged down at 500 g for 5 min and incubated at 37 C for 4 hours. Cells are resuspended by flicking the tube and incubated with CD11b-APC, CD14-APC, and CD66-PE at recommended dilutions for 45 min, protected from light. Cells are washed 2× with PBS, and supernatant is aspirated. 1% of PFA is added to the wells and stored at 4C until analysis.

T cell Cytotoxicity Assay. Effector (CD8+) cells are prepared by removing dead cells by density gradient centrifugation and resuspending in culture media. Target cells are prepared by removing dead cells by density gradient centrifugation and resuspending in correct density. Cells are resuspended in culture media with Cell Tracker Deep Red at 1:1000 for 30 min at 37C. Cells are washed and resuspended in culture media. A coculture is set up. Cells are seeded at desired effector: target ratio. Coculture time points desired for testing are set up. Ensure to have a well containing only labeled target cells to control for spontaneous target cell death. At each desired incubation time point, cells are collected, pelleted and resuspended in annexin V binding buffer mixed with annexin V PE and 7-AAD. Cells are incubate for 15 min at RT and labeled cells are analyzed by flow cytometry within one hour of staining.

Air Liquid Interface Immune Organoids. Permeable, membranous supports were inserted into cell culture plates to create a compartmentalized cell culture system like a transwell. Gel matrices were prepared by mixing collagen matrix with 10× concentrated sterile culture medium (Ham's F12) and sterile reconstitution buffer (2.2 g NaHCO₃ in 100 ml of 0.05 N NaOH and 200 mM HEPES) on ice at a 8:1:1 ratio until use. Reconstitution buffer was added and mixed to avoid bubbles. 1 ml of reconstituted collagen solution was added to the plate and was let to solidify by placing in a 37C incubator for 30 minutes. 1×10\hat{ }6−2×10\hat{ }6 cells were seeded into upper chamber of plates on top of the collagen matrix. Cmplete medium was added to the cells so that liquid height matches the height of the cells. Medium was replaced when color appeared to be changing.

Automated Image Segmentation for Area and Diameter. Brightfield image was opened and its histogram equalized. The portion of saturated pixels was set to 0%. Canny edge detection was performed with Gaussian kernel radius pixel resolution set according to setup. Maximum filter was run with radius set to value according to setup. This filter creates a running window that replaces the central pixel with the maximum value of the neighboring pixels. Morphological operation Closing was performed with number of iterations set to 10 and count set to 3. This filled the remaining small holes in the image. Morphological Opening was run with number of iterations set to 10 and count set to 3. This eliminated small structures like debris localized outside the organoids. The outline of segmented areas was overlayed with the original brightfield image and the precision of segmentation was checked. If necessary, the parameters of procedure was adjusted, focused especially on High threshold setting of Canny edge detection. Area was calculated using Analyze>Measure. Diameter was calculated using the formula d=21 (A/T).

Sandwich Antigen-Specific IgG ELISA. On the day of sample transfer, the plates containing the supernatant were thawed by placing them on ice for a couple of hours. Assay Diluent was made which is also the Blocking Buffer (1% BSA in 1×PBS w/o+Mg and +Ca). 4 ml of 7.5% BSA was added to 26 ml of DPBS (total=30 ml). Assay Diluent was stored at 4° C. for short term storage and −20° C. for long term storage. Wash Buffer (1×PBS+0.05% Tween-20) was made. 10×wash buffer (10×PBS+0.5% Tween-20) was prepared by adding 2.5 ml of Tween-20 into 500 ml of 10× PBS. 900 ml of MiliQ water was added to 100 ml of 10×buffer to prepare 1 1 of 1×Wash Buffer. Any remaining 10×wash buffer was stored at 4° C. for later use.

Coat ELISA Plate. Coating solution (1 μg/ml antibody in 1× Coating Buffer) was prepared as follows, ensuring a total volume of 100 μl/well. 5× ELISA coating buffer was dilauted to 1× working solution in DI water. Capture antibody was added to a final concentration of 1 μg/ml. 100 μl/well was added. The plate was covered with film and incubated on shaker with gentle shaking for 1 hr at 20−25° C. or overnight at 4° C.

Block ELISA Plate. The plate was washed as follows. Wash buffer was loaded into the plate washer. The plate was washed 4 times with 250 μl/well of wash buffer. The plate was blocked with 200 μl/well of Assay Diluent. The plate was covered with film and incubated on shaker at room temperature for 1 hour or overnight at 4° C.

Capture antigen/antibody. The plate was washed 4 times with 250 μl/well of wash buffer. The capture antigen/antibody solution was prepared as follows. Enough solution was prepared to ensure a total volume of 100 μl per well. The capture antigen/antibody was added in assay diluent to achieve a final concentration of 1 μg/ml. The plate was coated with 100 μl/well of the prepared antigen/antibody solution to the plate. The plate was covered and incubated on a shaker at room temperature for 1 hour.

Intracellular Staining. 6 mL Fixation/Permeabilization working solution was prepared as follows. 1.5 mL Foxp3 Fixation/Permeabilization Concentrate was added to 4.5 mL Foxp3 Fixation/Permeabilization Diluent. 40 mL 1×Permeabilization Buffer was prepared as follows. 4 mL 10×Permeabilization Buffer was added to 36 mL MilliQ water. Disaggregated organoid cells were added to two separate tubes containing 9 mL cell culture medium and were centrifuged (5 min, 350 g). Supernatant was decanted. 2E6 cells was transfered to two 1.5 mL tubes per donor (Tube 1: Live/Dead only, fixed, Tube 2: Full panel, fixed) and centrifuged (5 min, 350 g). Supernatant was flicked to remove. Cells were resuspended in 200 μL Live-Dead Aqua diluted 1:500 in PBS and incubated 15 minutes away from light at RT. 2 μL of each surface stain was added from claim 16/18 panel to 188 μL PBS+05% BSA (CD20-Pacific Blue, CD3-FITC, CXR4-PerCP-Cy5.5, IgD-PE, CD83-APC, CD45-AF700). When cells were finished incubating, surface stain cocktail was added directly to Tube 2. 200 uL PBS+0.5% BSA was added to Tube 1. Cells were incubated 15 minutes away from light at RT. When cells were finished incubating, 200 uL PBS+0.5% BSA was added and spun (5 min, 350 g). Supernatants were flicked to remove. 400 μl PBS+0.5% BSA was added and spun again (5 min, 350 g). Supernatants were flicked to remove. This step was then repeated. 1 mL of Foxp3 Fixation/Permeabilization working solution was then added to each tube, pulse vortexed, and incubate for 30 minutes at 4C. Cells were transferred to two 15-mL Falcon tubes. 2 mL 1× Permeabilization Buffer was added to both tubes and centrifuged (5 min, 350 g). Supernatant was flicked and cells were resuspended in residual Permeabilization Buffer. 2 μL of Ki67-BV605 was added to resuspended cells and mixed by pipetting. This step was repeated. Supernatant was flicked, cells were resuspended in 250 uL PBS+0.5% BSA, and then run on flow.

Sandwich Antigen-Specific IgM ELISA. ELISA. On the day of sample transfer, the plates containing the supernatant were thawed by placing them on ice for a couple of hours. Assay Diluent was made which is also the Blocking Buffer (1% BSA in 1×PBS w/o+Mg and +Ca). 4 ml of 7.5% BSA was added to 26 ml of DPBS (total=30 ml). Assay Diluent was stored at 4° C. for short term storage and −20° C. for long term storage. Wash Buffer (1×PBS+0.05% Tween-20) was then made. 10× wash buffer (10×PBS+0.5% Tween-20) was prepared by adding 2.5 ml of Tween-20 into 500 ml of 10× PBS. 900 ml of MiliQ water was added to 100 ml of 10× buffer to prepare 1 1 of 1×Wash Buffer. Any remaining 10× wash buffer was stored at 4° C. for later use.

Coat ELISA Plate. Coating solution (1 ug/ml antibody in 1× Coating Buffer) was prepared as follows, ensuring a total volume of 100 μl/well. 5× ELISA coating buffer was diluted to 1× working solution in DI watert. Capture antibody was added to a final concentration of 1 μg/ml and 100 μl/well was added. The plate was covered with film and incubated on shaker with gentle shaking for 1 hr at 20−25° C. or overnight at 4° C.

Block ELISA Plate. Wash buffer was loaded into the plate washer, and the plate was washed 4 times with 250 μl/well of wash buffer. The plate was blocked with 200 μl/well of Assay Diluent. The plate was covered with film and incubated on shaker at room temperature for 1 hour or overnight at 4° C.

The plate was then washed 4 times with 250 μl/well of wash buffer. The capture antigen/antibody solution was prepared as follows. Enough solution was prepared to ensure a total volume of 100 μl per well. The capture antigen/antibody was added in assay diluent to achieve a final concentration of 1 μg/ml. The plate was coated with 100 μl/well of the prepared antigen/antibody solution to the plate. The plate was covered and incubated on a shaker at room temperature for 1 hour.

### Example 2: The Stem Cell Population is Represented in Immune Organoids

This example demonstrates that immune organoids contain stem cell populations only found in secondary lymphoid organs and not blood. These stem cells proliferate and play a role in lymph node support and function as well as immune organoid support and function.

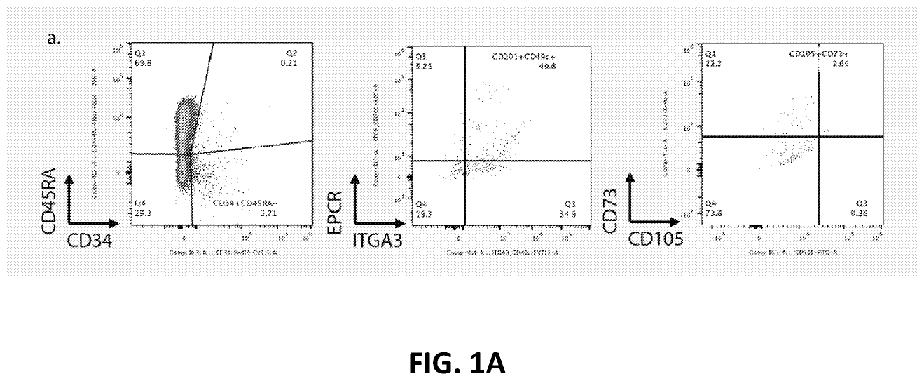

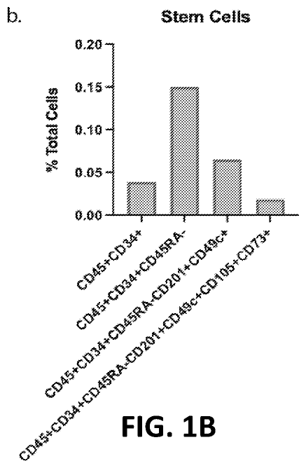

Briefly, immune organoids were generated using the methods described in Example 1. Day 7 cultures were stained for stem cell markers, and flow cytometry was performed. As shown in FIG. 1A and FIG. 1B, day 7 immune organoids contain populations of CD45+CD34+, CD45+CD34+CD45RA−, CD45+CD34+CD45RA-CD201 (EPCR)+CD49c (ITGA3)+, CD45+CD34+CD45RA-CD201 (EPCR)+CD49c (ITGA3)+CD105+CD73+ stem cells.

### Example 3: Diverse Immune Cell Populations are Contained in a Human Immune Organoid, Including Both Innate and Adaptive Cells

This example demonstrates that immune organoids are composed of the same diverse immune cell types as those found in a human lymph node. These cells work together to recapitulate lymph node structure and function.

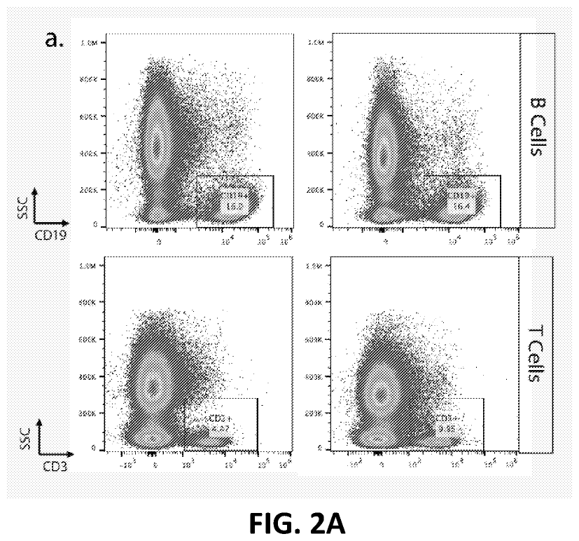

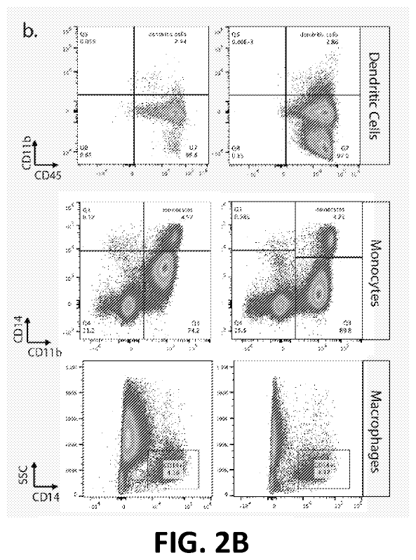

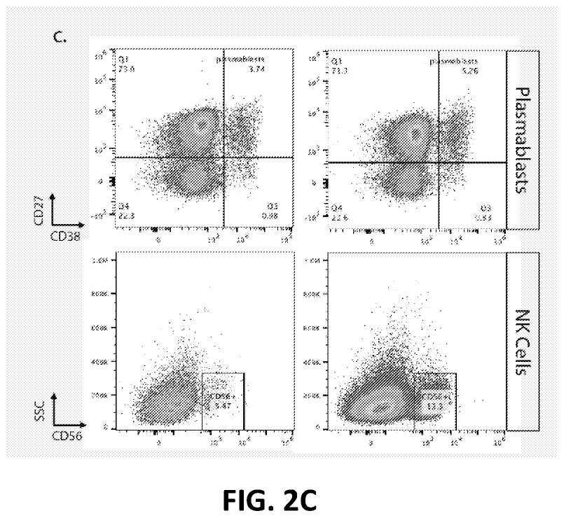

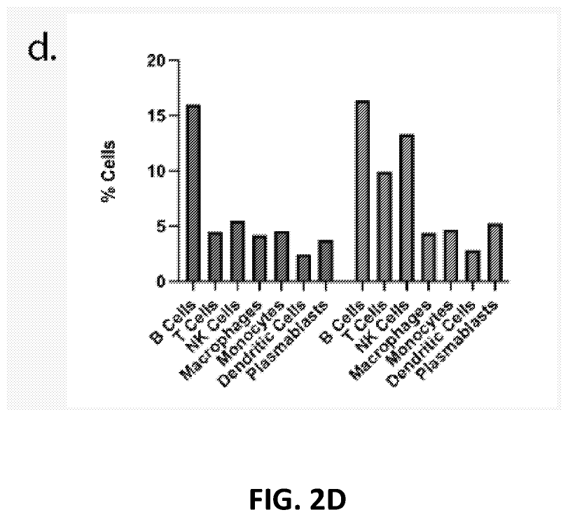

Briefly, immune organoids were generated using the methods described in Example 1. Day 7 cultures were stained for markers of B cells, T cells, NK cells, macrophages, monocytes, dendritic cells, and plasmablasts. As shown in FIG. 2A, B cells were identified as a CD19+ population, and T cells were identified as a CD3+ population. FIG. 2B shows the presence of CD11b+CD45+dendritic cells, CD11b+CD14+ monocytes, and CD14+macrophages. FIG. 2C shows the presence of CD38+CD27+plasmablasts, and CD56+NK cells. FIG. 2D shows the quantification of immune cell subsets in the day 7 immune organoid cultures.

### Example 4: Immune Organoids Undergo B Cell Differentiation Upon Stimulation

This examples demonstrates that immune organoids undergo a variety of different responses upon stimulation, including B cell differentiation.

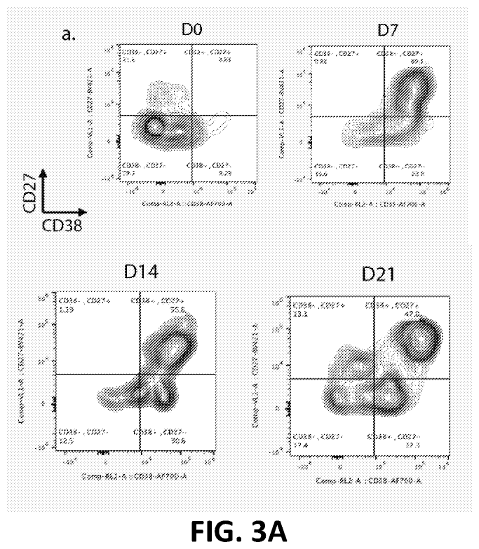

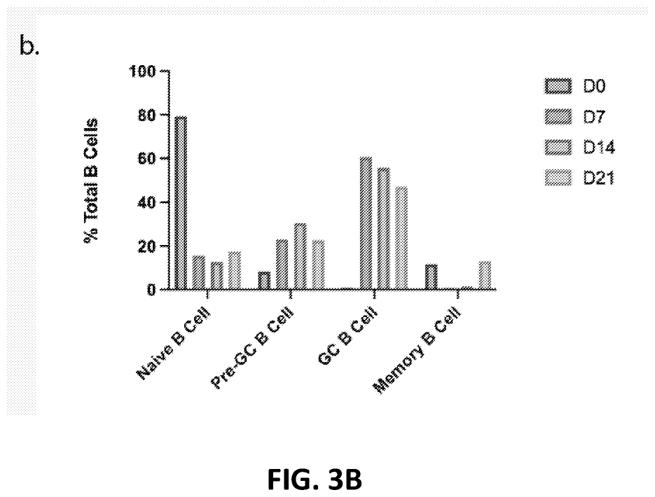

Briefly, immune organoids were generated using the methods described in Example 1. FIG. 3A shows cells previously gated on total B cells (CD3-CD19+CD45+), and then stained for CD38-CD27-naïve B cell, CD38-CD27+ memory B cell, CD38+CD27-pre-GC B cell, CD38+CD27+GC B cell. FIG. 3B shows immune organoids stimulated at day 0 are primarily naive B cells and then differentiate into pre-GC B cells, GC B cells, memory B cells, and plasmablasts. These B cell phenotypes are consistent with human B cell responses to infection or vaccination or other stimulation in a lymph node.

### Example 5: Immune Organoids are Composed of T Cell Subtypes

This examples demonstrates that immune organoids are composed of T cells, including T cell subtypes that are consistent with those found in human lymph nodes.

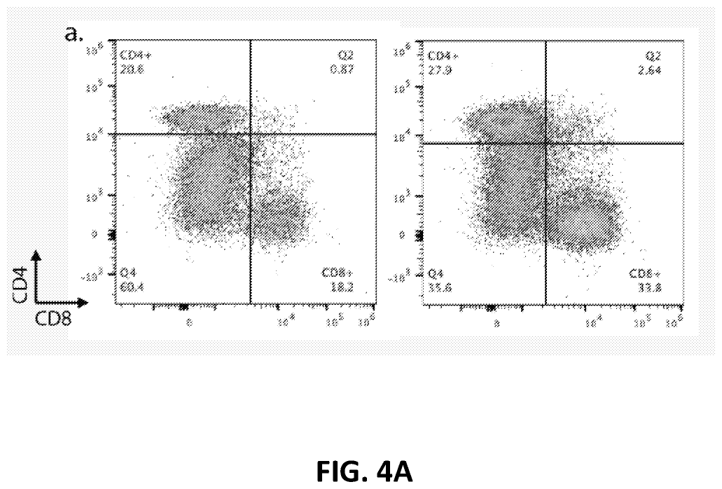

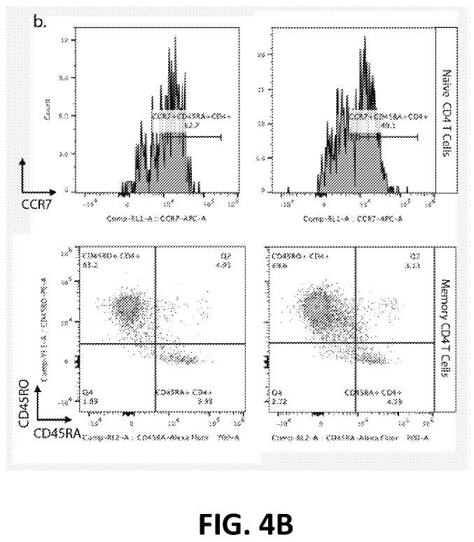

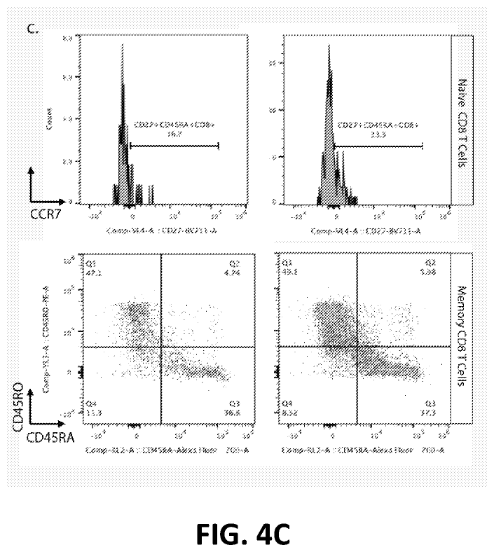

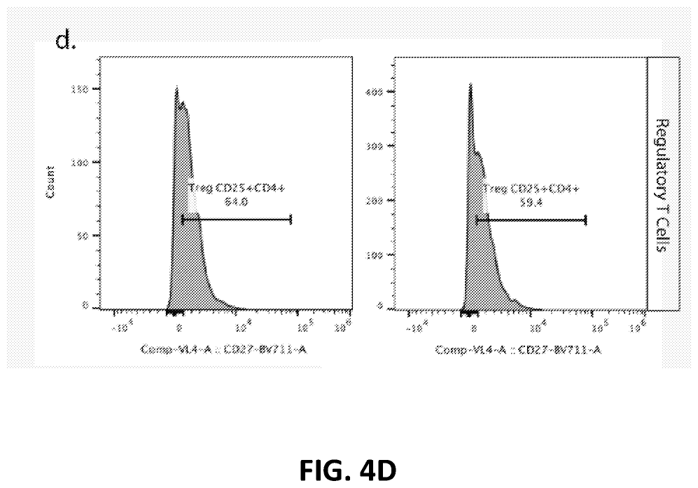

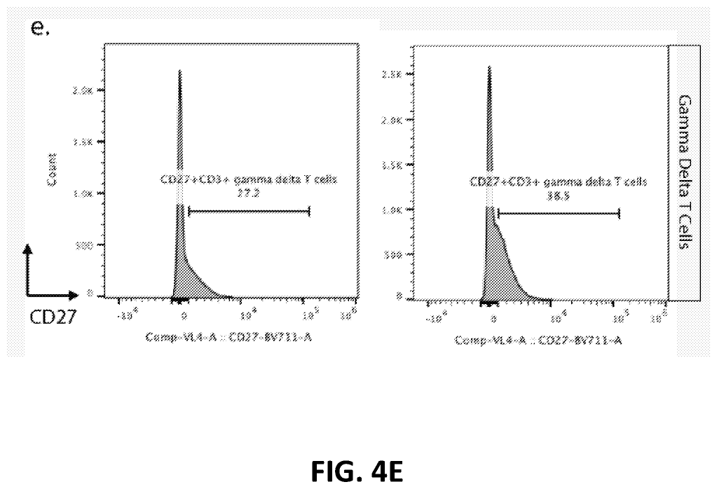

Briefly, immune organoids were generated using the methods described in Example 1. FIG. 4A shows day 7 cultures previously gated on total T cells (CD19-CD3+). Cells were then stained for CD4+ and CD8+ T cells by flow cytometry.

FIGS. 4B-4C show flow cytometry staining demonstrating immune organoids consist of T cell subtypes. All cells shown were previously gated on total T cells (CD19-CD3+) and then subsequently gated on CD4+ T cells and CD8+ T cells for their respective Naive and Memory T cell populations. Phenotypes shown: CD4+CCR7+CD45RA+Naive CD4 T cells, Naive CD8 T cells, CD4+CCR7+CD45RA+, CD4+CD45RA-CD45RO+Memory CD4 T cells, CD8+CD45RA-CD45RO+Memory CD8 T cells.

FIGS. 4D-4E show flow cytometry staining demonstrating immune organoids consist of additional T cell subtypes. All cells shown were previously gated on total T cells (CD19-CD3+). Cell types shown: CD3+CD4+CD25+ Tregs, CD3+CXCR5+CD25+ T follicular helper cells, CD3+CD27+gamma delta T cells.

### Example 6: Immune Organoids are Composed of Other Immune Cell Types

This example demonstrates that immune organoids are composed of rarer immune cell types critical for human lymph node.

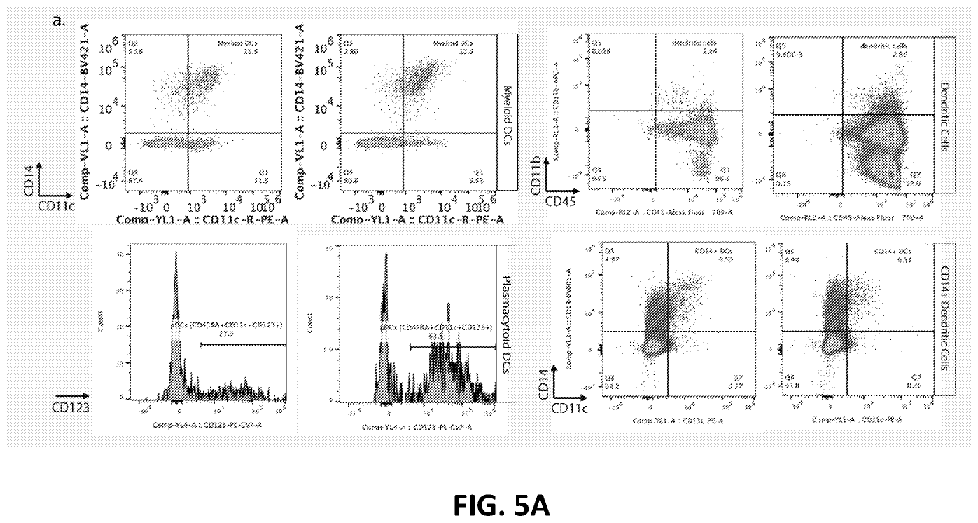

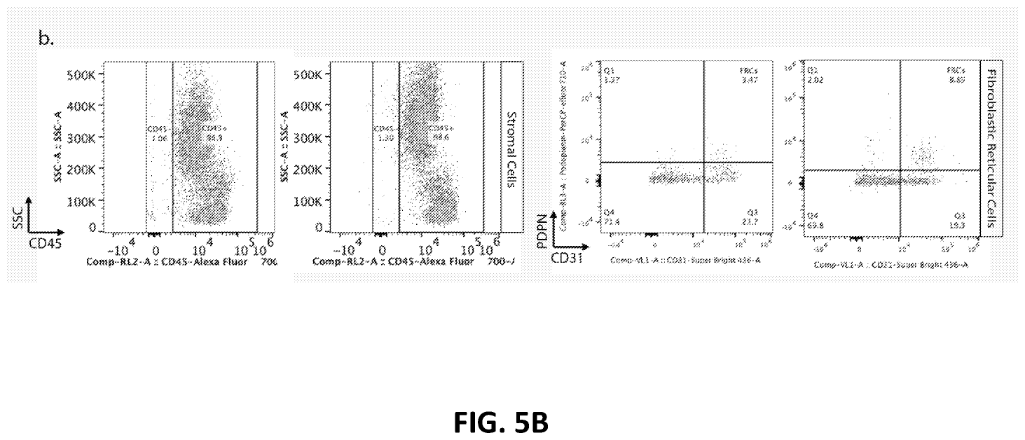

Briefly, immune organoids were generated using the methods described in Example 1. Day 7 cultures were stained for myeloid DCs (CD14+CD11c+), plasmacytoid DCs (CD123+), conventional DCs (CD11b+CD45+), and CD14+DCs (CD14+CD11c+) (FIG. 5A). As shown in FIG. 5B, day 7 cultures also include cell types such as stromal cells (CD45−) and fibroblastic reticular cells (CD31+PDPN+).

### Example 7: Size of Immune Organoids

This example demonstrates that immune organoid size is dependent upon the number of cells that are used to form organoids as well as dependent upon the stimulation conditions. Organoids grow considerably after successful stimulation due to cellular proliferation and activation.

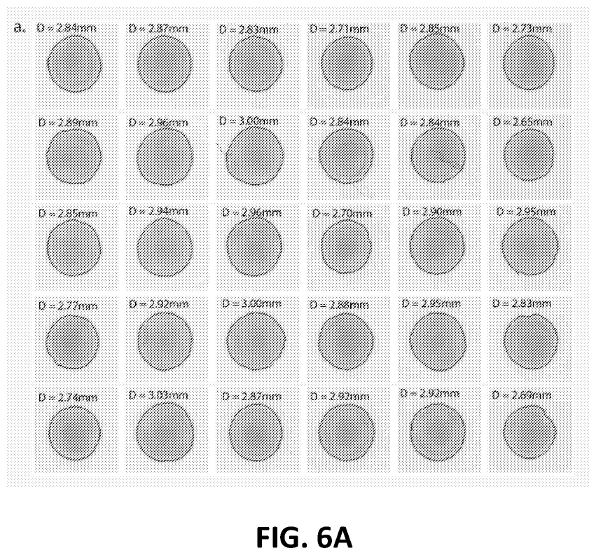

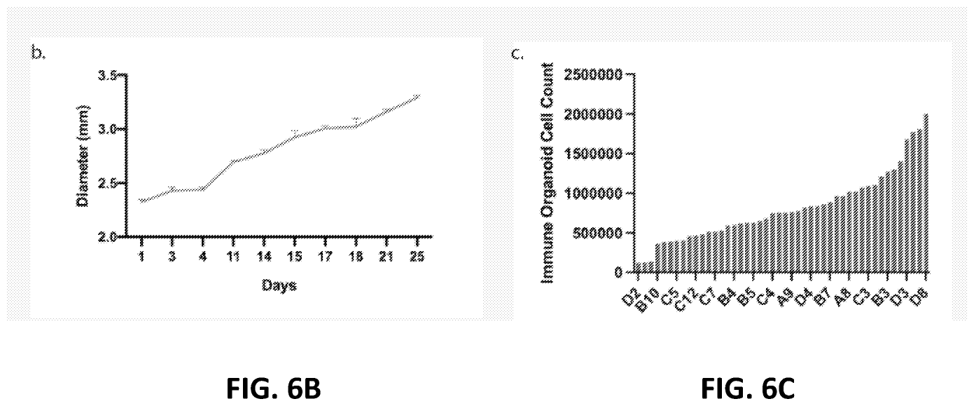

Briefly, immune organoids were generated using the methods described in Example 1. Day 14 cultures were imaged and diameter was measured as shown in FIG. 6A. As shown in FIG. 6B, the diameter of the organoids increases from day 1 to day 21. The total number of cells in the organoids also increases (FIG. 6C).

### Example 8: Immune Organoids Form Germinal Centers

This example demonstrates that germinal centers with light zones and dark zones are required to recapitulate lymph node biology and are unable to be found in current in vitro technologies. The organoid platform described herein uniquely produces germinal centers and thus reproduces a critical feature of human biology.

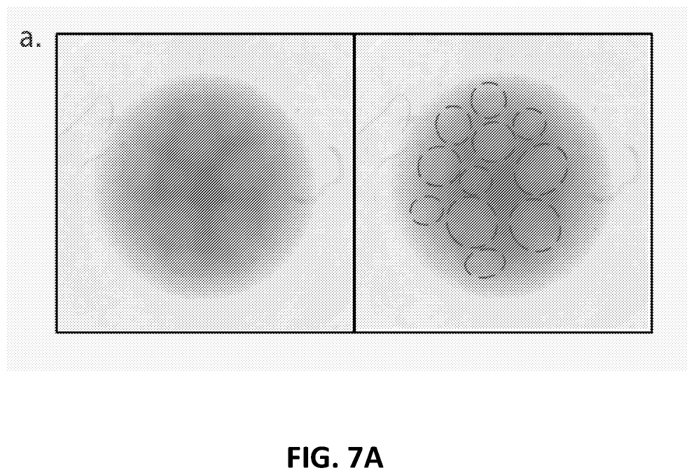

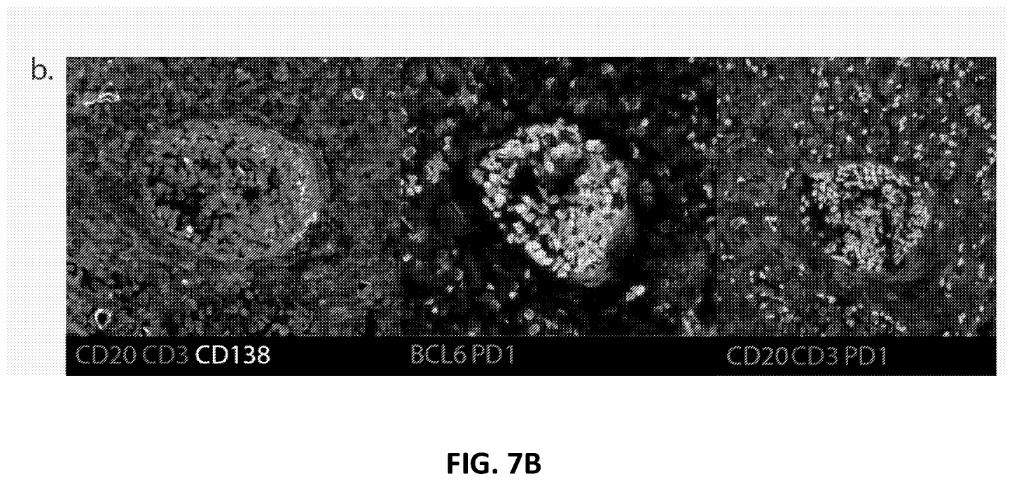

Briefly, immune organoids were generated using the methods described in Example 1. Day 14 cultures were stimulated with Hepatitis B vaccines and organoids were visualized under brightfield microscopy. As shown in FIG. 7A, lighter structures in the organoids outlined in red are consistent with germinal center morphology.

Confocal microscopy was then performed to image the germinal centers in the day 14 immune organoids with B (CD20) and T cell (CD3) organization, plasmablasts (CD138), BCL6+ cells, and PD1+ cells (FIG. 7B).

### Example 9: Immune Organoids have Cells Consistent with Germinal Center Function

This example demonstrates that germinal centers need to be present as well as functional. These data indicate that these germinal centers are fully functional and reproduce all essential aspects of lymph node function, being the first organoid technology capable of doing so.

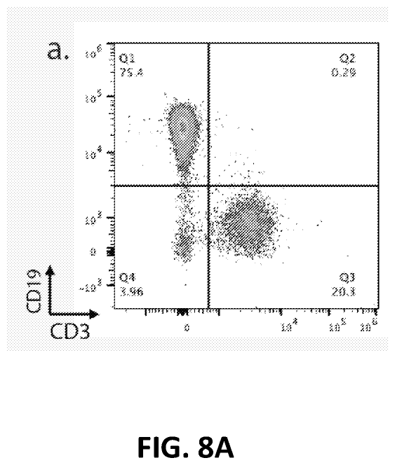

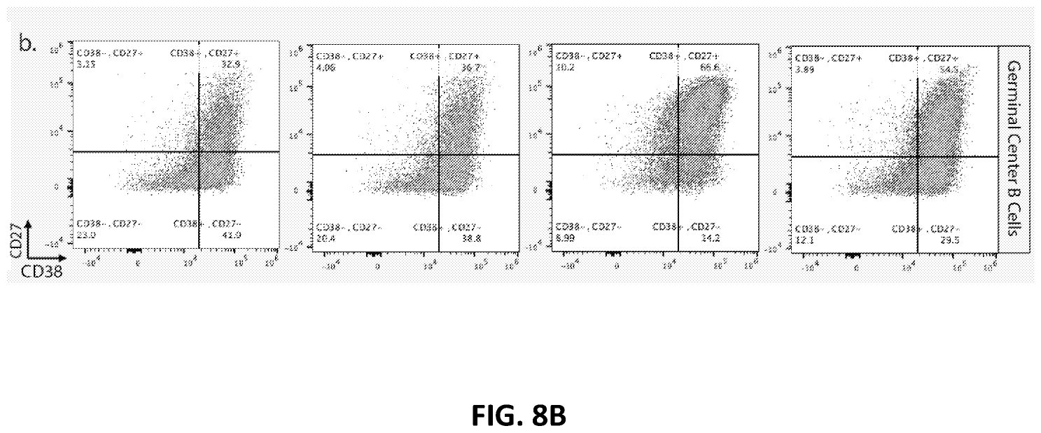

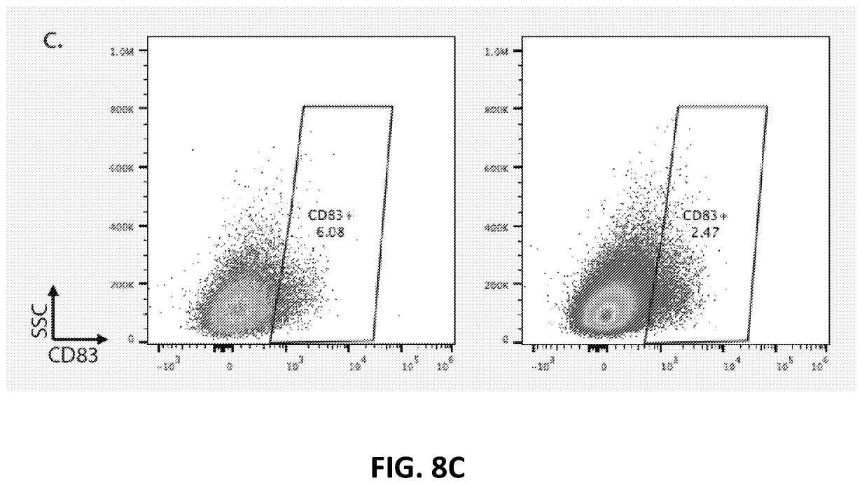

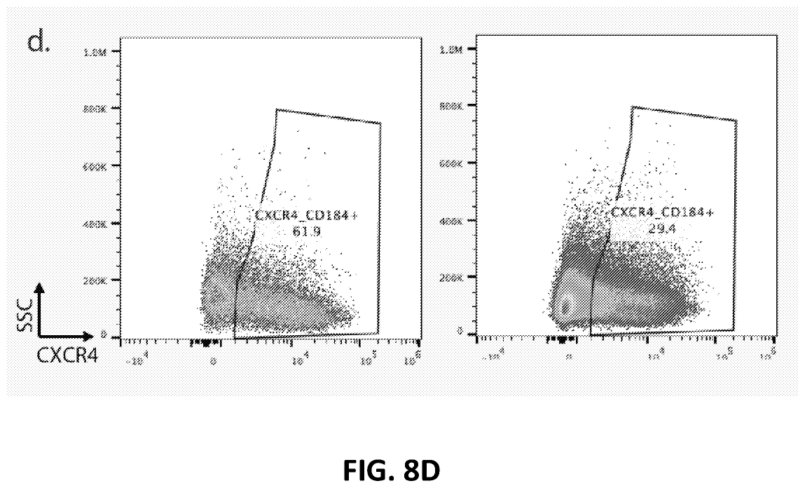

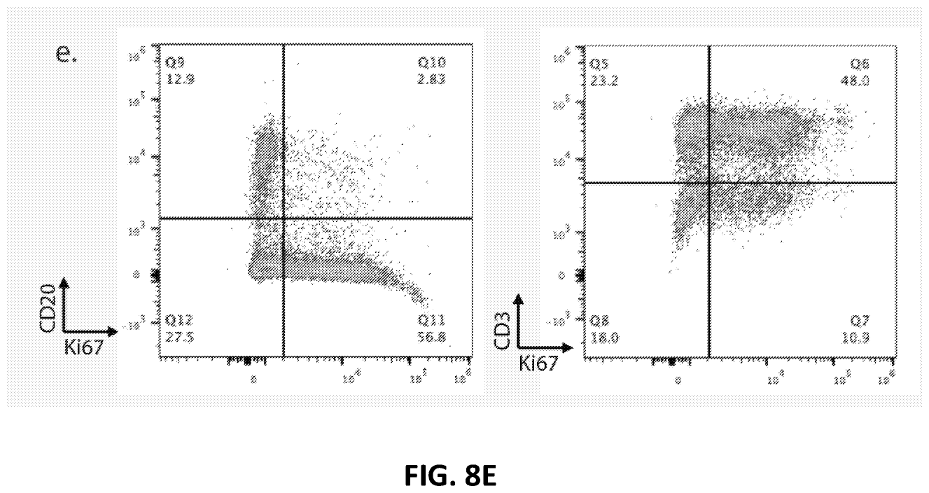

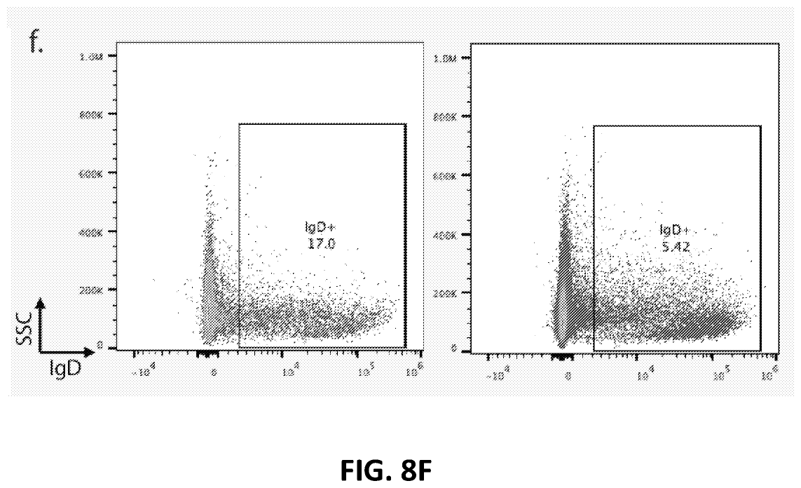

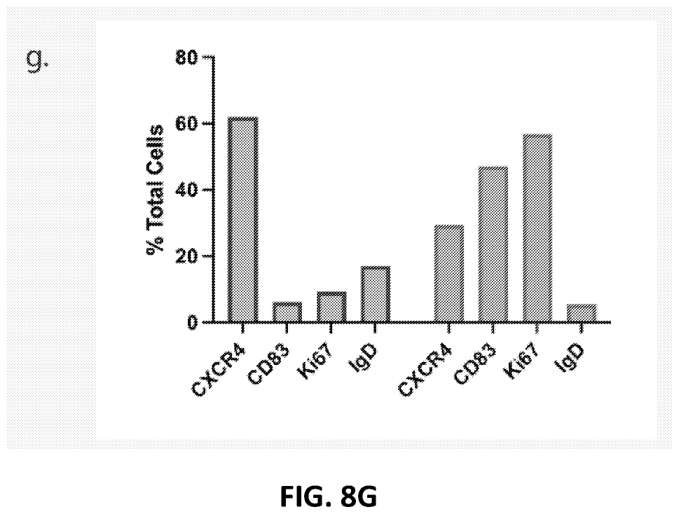

FIG. 8A shows flow cytometry staining demonstrating immune organoids consisting of B and T cell zones. Cells shown CD19+ B cells and CD3+ T cells. Plots are from representative day 14 immune organoids. Flow cytometry was then performed to identify germinal center B cells. As shown in FIG. 8B, day 14 cultures contain CD27+CD38+germinal center B cells.

Additonal cell types were then identified in day 14 cultures, as shown in FIGS. 8C-8F. In particular, day 14 immune organoids consist of cells found in germinal centers including CD83+, Ki67+ B cells, Ki67+ T cells, IgD+ cells, and CXCR4+ cells. Cell types were quantified as shown in FIG. 8G.

### Example 10: Immune Organoids are Composed of Both B and T Cells that can be Modulated Upon Various Stimulation Conditions

This example demonstrates that immune organoids are composed of a large mixture of different immune cells that are activated and can proliferate upon stimulation. B and T cells are one example of dramatic differences in cell abundance after stimulating the immune organoids with six different stimulation conditions, where some stimulation conditions preferentially expanded B cell populations and some expanded T cell populations.

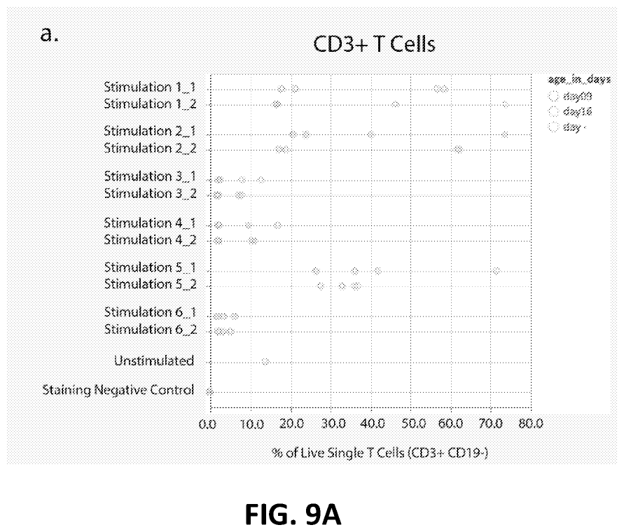

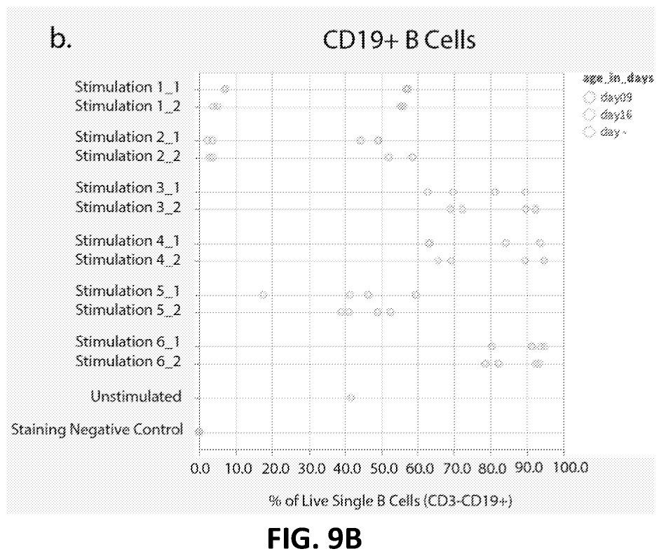

Briefly, immune organoids were generated using the methods described in Example 1. As shown in FIGS. 9A-9B, day 9 and day 16 cultures were stimulated across different conditions. The stimulations were protein stimulations for influenza (hemagluttinin protein in this case) with different combinations of adjuvants inportated across all 6 different conditions. The frequencies of T (FIG. 9A) and B (FIG. 9B) cells were determined by flow cytometry.

### Example 11: Immune Organoids Undergo a Full Adaptive Immune Response

This example demonstrates that organoids produce plasmablasts and antigen-specific antibodies against targets to which patient donors have been exposed (recall response) and to which patients are naïve. The antibodies produced by the organoids have the ability to class switch, for example IgM to IgG antibodies, consistent with an adaptive immune response.

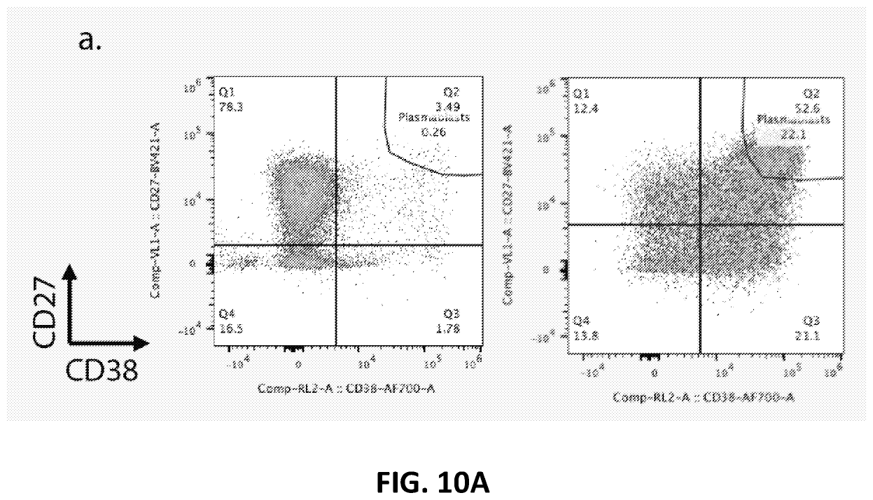

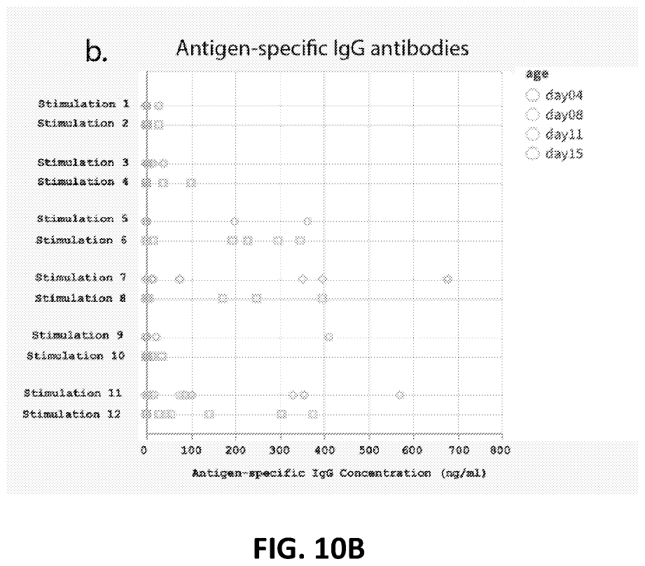

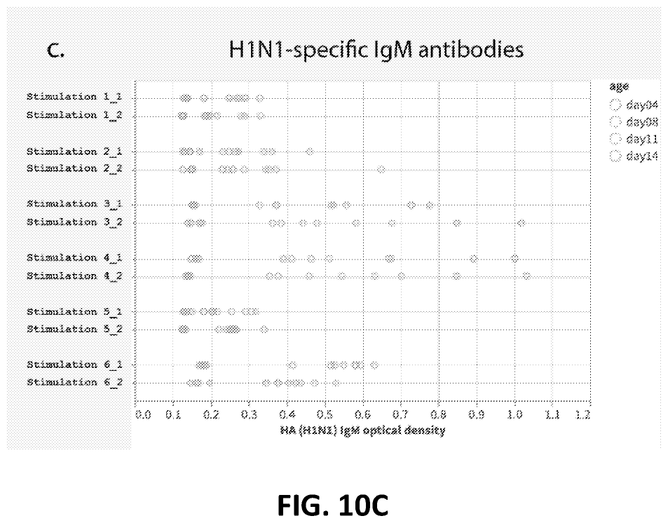

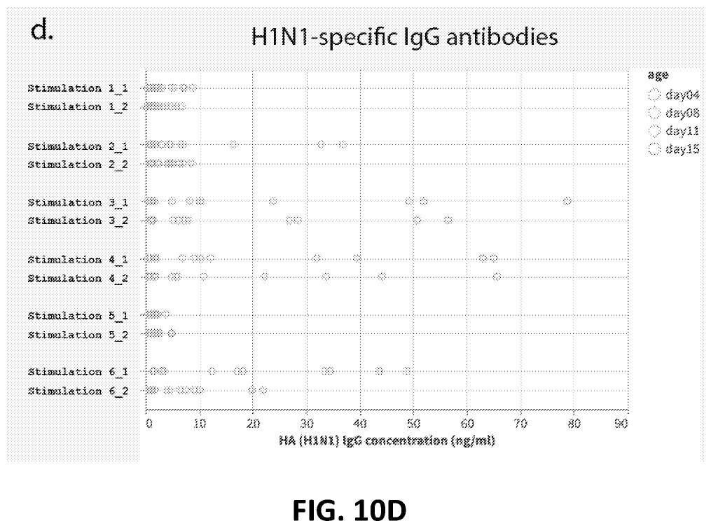

Briefly, as shown in FIG. 10, upon stimulation, immune organoids undergo a full immune response including a full adaptive immune response. This includes full B cell differentiation from naive B cells into germinal center B cells and mature cells like memory B cells and plasmablasts. Upon induction of plasmablasts, these cells then produce antigen-specific antibodies against targets to which patients have been stimulated with. The immune organoids will produce antibodies against targets to which patients have been previously exposed in a recall response as well as targets to which the patients are completely naive in a naive response. The antibodies produced by the organoids are antigen-specific and have the ability to class switch, for example IgM to IgG antibodies, consistent with an adaptive immune response.

### Example 12: Immune Organoids can Break Tolerance to Generate Antibodies Against Human Targets

This example demonstrates that immunotolerance is a key attribute of systemic immunity, as is being able to model breaking tolerance (i.e. autoimmune disease and allergic disease). No in vitro platform is currently capable of doing this in a way that mimics human patients. These data indicate that the organoids described herein produce autoimmune responses against self antigen, key to being able to model these diseases.

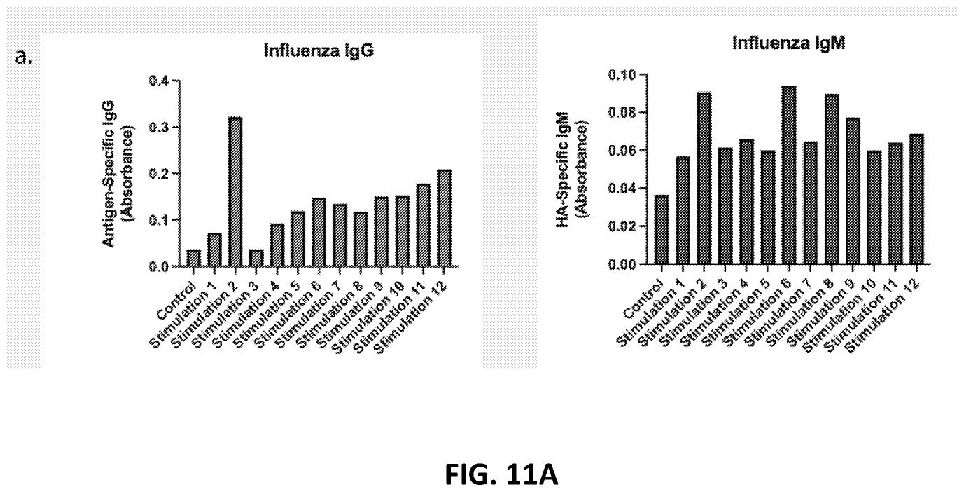

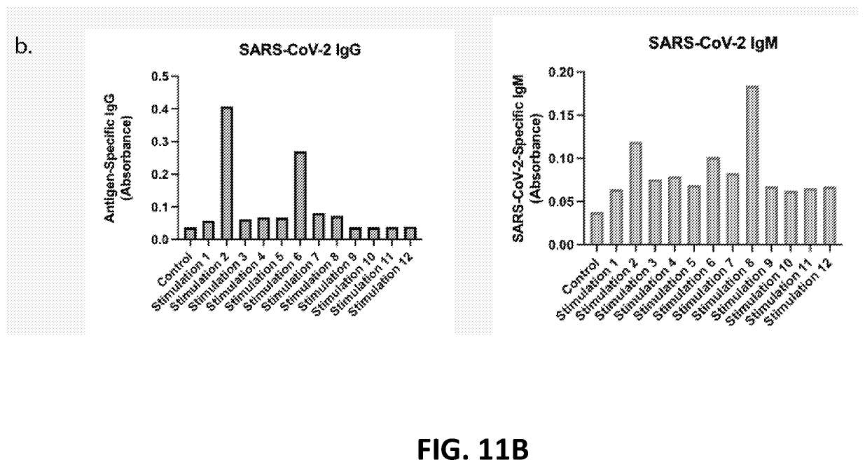

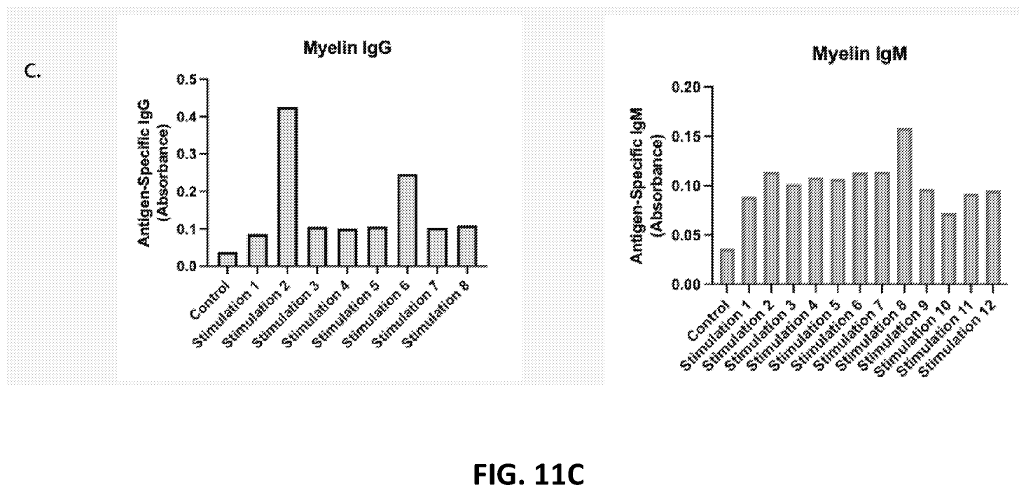

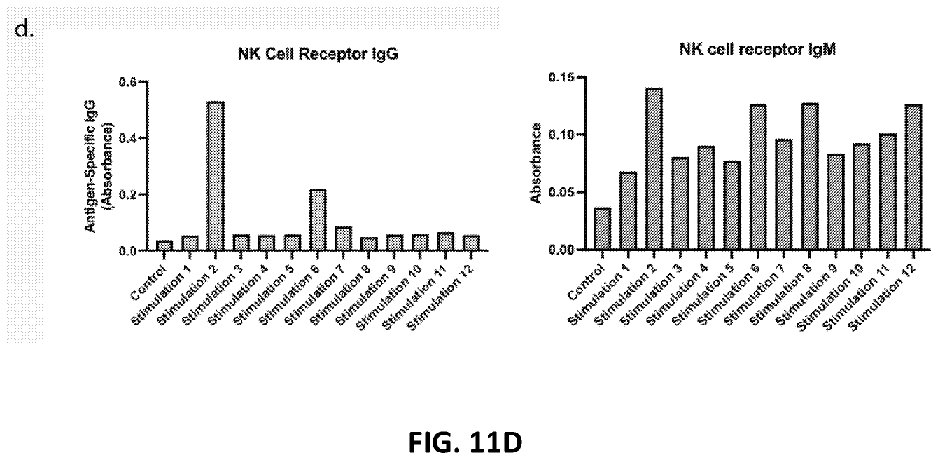

Briefly, as shown in FIGS. 11A-11D, the immune organoids produce both IgM and IgG antibodies against foreign antigens, examples here include influenza and SARS-COV-2. Immunotolerance is a key attribute of systemic immunity, in which immune organoids replicate this feature by not responding to stimulation. Another key attribute of systemic immunity is the ability to break tolerance (i.e. autoimmune disease and allergic disease). Under certain stimulation conditions, the immune organoids are able to model breaking tolerance, a key feature for the immune organoid to produce antibodies against human targets for therapeutic purposes (i.e. cancer and autoimmune disease) and the organoid has the ability to produce autoimmune responses against self antigen (myelin) and are key to being able to model autoimmune and allergic diseases for the first time.

### Example 13: Immune Organoids Form within 24 Hours and Remain Viable for at Least 30 Days

This example demonstrates the ability of the organoids to form their essential structures and begin function after one day. These organoids currently remain viable in culture for 30 or more days.

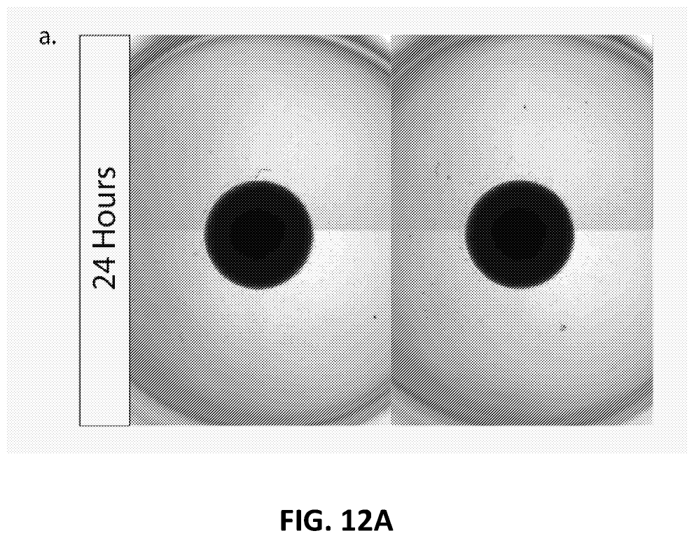

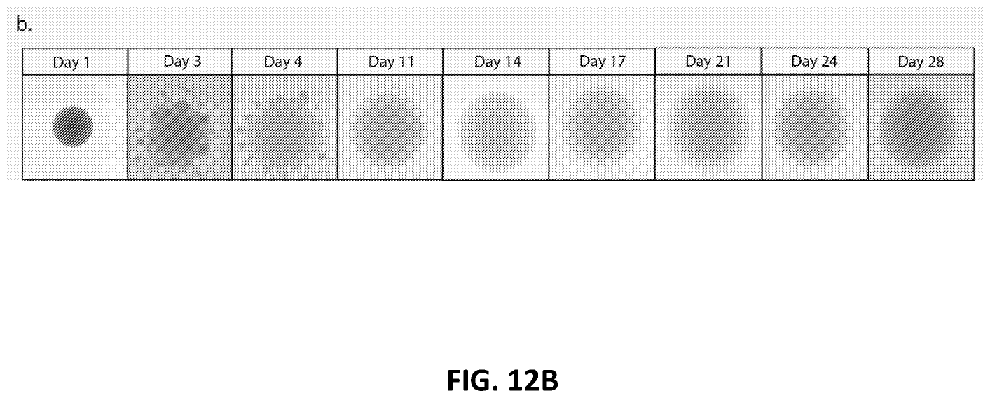

Briefly, immune organoids were generated using the methods described in Example 1. Organoids were visualized using brightfield microscopy after 24 hours (FIG. 12A) and then through day 28 (FIG. 12B).

### Example 14: Immune Organoids can also be Cultured at the Air-Liquid-Interface

This example demonstrates there are numerous methods for organoid manufacture including but not limited to air liquid interface (ALI), scaffolds, 3D printed plates, and hydrogels. These have been tested methods developed to produce these organoids in suspension and in ALI.

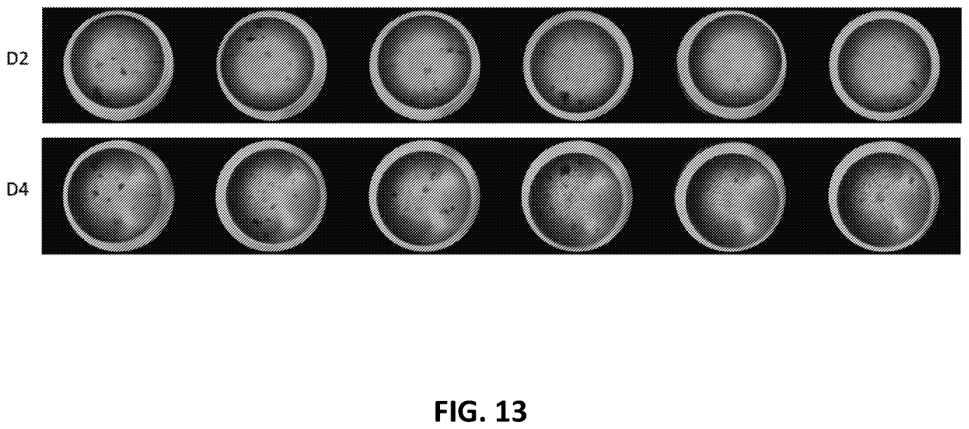

Briefly, there are numerous methods for organoid manufacture including but not limited to air liquid interface (ALI), scaffolds, 3D printed plates, and hydrogels. We have tested all of these methods on the immune organoids, and as shown in FIG. 13, we have developed methods to produce these organoids in suspension and using the ALI method, a common method of organoid production. This is a method by which organoids are seeded on solid pedestals (i.e. collagen) to raise the organoids so that they are cultured at the surface of the medium, leaving much of the organoid submerged but having the top of it exposed to air. There are several benefits to this method including increasing oxygen penetration into the organoids as well as using liquid to modulate organoid morphology and height for interesting functions and improved imaging capabilities. This is an additional method for which immune organoids can be made.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

What is claimed is:

1. A three dimensional immune organoid comprising a plurality of self-assembled primary immune cells obtained from one or more secondary lymphoid organs and a plurality of stem cells, wherein said stem cells are CD34+, CD45RA−, ITGA3+, EPCR+, CD90+, CD73+, and CD105+.

2. The immune organoid of claim 1, wherein the one or more secondary lymphoid organs are from spleen, lymph node, Peyer's patch, and MALT.

3. The immune organoid of any preceding claim, wherein the immune organoid is a human immune organoid.

4. The immune organoid of any preceding claim, further comprising peripheral blood mononuclear cells.

5. The immune organoid of any preceding claim, wherein the plurality of immune cells is obtained from living patients, surgical resections, fine needle aspirates, biopsy, and deceased patients.

6. The immune organoid of any preceding claim, wherein the plurality of immune cells comprises B cells, T cells, plasmablasts, NK cells, monocytes, dendritic cells, macrophages and combinations thereof.

7. The immune organoid of claim 6, wherein the B cells comprise one or more naïve B cells, pre-GC B cells, GC B cells, memory B cells, or a combination thereof.

8. The immune organoid of claim 6, wherein the T cells comprise naïve CD4 T cells, memory CD4 T cells, T regulatory cells, T follicular helper cells, naïve CD8 cells, memory CD8 cells, gamma delta T cells, or a combination thereof.

9. The immune organoid of claim 6, wherein the dendritic cells comprise conventional dendritic cells, plasmacytoid dendritic cells, myeloid dendritic cells, or a combination thereof.

10. The immune organoid of any preceding claim, wherein the plurality of primary immune cells further comprises one or more stromal cells and fibroblastic reticular cells.

11. The immune organoid of any preceding claim, wherein the immune organoid is 8000 μm or less in diameter.

12. The immune organoid of any preceding claim, wherein the immune organoid comprises germinal centers and/or B/T cell zones.

13. The immune organoid of claim 12, wherein the immune organoid is CXCR4⁺, CD83⁺, Ki67⁺, and IgD⁺.

14. The immune organoid of claim 12, wherein the immune organoid is CD3⁺ and CD20⁺.

15. The immune organoid of any preceding claim, wherein the immune organoid produces antibodies.

16. The immune organoid of claim 15, wherein the antibodies have full humoral functionality.

17. The immune organoid of claim 16, wherein the antibodies bind human and non-human targets.

18. The immune organoid of claim 17, wherein the human targets comprise proteins, sugars, and nucleic acid.

19. The immune organoid of claim 17, wherein the non-human targets comprise infectious disease antigens, venoms, poisons, small molecules.

20. A composition comprising:
a plurality of primary immune cells of which 100% self-assemble into 3 dimensional immune organoids within 24 hours.

21. The composition of claim 20, wherein the plurality of immune cells are obtained from one or more secondary lymphoid organs.

22. The composition of claim 21, wherein the one or more secondary lymphoid organs are from spleen, lymph node, Peyer's patch, and MALT.

23. The composition of any one of claims 20-22, wherein the plurality of immune cells are human immune cells.

24. The composition of any one of claims 20-23, wherein the plurality of immune cells comprises 2×10⁶ or fewer cells.

25. The composition of any one of claims 20-24, further comprising peripheral blood mononuclear cells.

26. The composition of any one of claims 20-25, wherein the plurality of immune cells is obtained from living patients, surgical resections, fine needle aspirates, biopsy, and deceased patients.

27. The composition of any one of claims 20-26, wherein the plurality of immune cells comprises B cells, T cells, plasmablasts, NK cells, monocytes, dendritic cells, macrophages and combinations thereof.

28. The composition of claim 27, wherein the plurality of primary immune cells further comprises one or more stromal cells, fibroblastic reticular cells, and stem cells.

29. A method of producing multiple three dimensional immune organoids, the method comprising:
(a) dissociating tissue from one or more secondary lymphoid organs to produce a plurality of single primary immune cells;
(b) contacting 1×10⁶ or fewer of the plurality of single primary immune cells with a solid support; and
(c) culturing the plurality of single primary immune cells for 24 hours to produce multiple three dimensional immune organoids, wherein the multiple immune organoids remain viable for at least 30 days.

30. The method of claim 29, wherein the method comprises freezing the plurality of single primary immune cells prior to (b).

31. The method of claim 30, wherein the method comprises thawing the plurality of single primary immune cells and treating with a ROCK inhibitor after freezing.

32. The method of any one of claims 29-31, wherein the immune organoid is a human immune organoid.

33. The method of any one of claims 29-32, wherein secondary lymphoid organs are spleen, lymph node, Peyer's patch, and MALT.

34. The method of any one of claims 29-33, wherein the secondary lymphoid organs are obtained from living patients, surgical resections, fine needle aspirates, biopsy, and deceased patients.

35. The method of any one of claims 29-34, wherein the plurality of primary immune cells comprises B cells, T cells, plasmablasts, NK cells, monocytes, dendritic cells, macrophages and combinations thereof.

36. The method of any one of claims 29-35, wherein the plurality of primary immune cells comprises one or more stromal cells, fibroblastic reticular cells, and stem cells.

37. The method of any one of claims 29-36, wherein the immune organoid is 8000 μm or less in diameter.

38. The method of any one of claims 29-37, wherein the immune organoid is a 3 dimensional structure.

39. The method of any one of claims 29-38, wherein the immune organoid comprises germinal centers and/or B/T cell zones.

40. The method of claim 39, wherein the immune organoid is CXCR4⁺, CD83⁺, Ki67⁺, and IgD⁺.

41. The method of claim 39, wherein the immune organoid is CD3⁺ and CD20⁺.

42. The method of any one of claims 29-41, wherein the immune organoid produces antibodies.

43. The method of claim 42, wherein the antibodies have full humoral functionality.

44. The method of claim 42, wherein the antibodies bind human and non-human targets.

45. The method of claim 44, wherein the human targets comprise proteins, sugars, and nucleic acid.

46. The method of claim 44, wherein the non-human targets comprise infectious disease antigens, venoms, poisons, small molecules.
